(19) United States
(12) Patent Application Publication
(10) Pub. No.: US 2025/0356511 A1
(43) Pub. Date: Nov. 20, 2025

(54) SYSTEMS AND METHODS FOR REAL-TIME MULTIMODAL DEFORMABLE IMAGE REGISTRATION FOR IMAGE-GUIDED INTERVENTIONS

(71) Applicants: GE Precision Healthcare LLC, Waukesha, WI (US); Regents of the University of Michigan, Ann Arbor, MI (US); University of Iowa Research Foundation, Iowa City, IA (US)

(72) Inventors: Jhimli Mitra, Niskayuna, NY (US); Chitresh Bhushan, Schenectady, NY (US); Soumya Ghose, Niskayuna, NY (US); Desmond Teck Beng Yeo, Clifton Park, NY (US); Thomas Kwok-Fah Foo, Clifton Park, NY (US); Shane Wells, Madison, WI (US); Jim Holmes, Solon, IA (US)

(21) Appl. No.: 18/669,416

(22) Filed: May 20, 2024

(51) Int. Cl. G06T 7/33; G06T 3/18

(52) U.S. Cl. CPC G06T 7/344; G06T 3/18; G06T 2207/10088; G06T 2207/10136; G06T 2207/20081; G06T 2207/30004

(57) ABSTRACT

Systems and methods are provided for real-time multimodal deformable image registration for image-guided interventions. A pre-interventional three-dimensional (3D) magnetic resonance imaging (MRI) image and multiple 3D ultrasound (US) images capturing various respiratory states and poses are acquired for a patient. The MRI image is registered to each US image using a trained MR-US deformation model, producing deformed MRI images. During intervention, an interventional 3D US image is acquired and registered to a pre-interventional US image using a trained US-US deformation model, determining a warp field. This warp field is applied to the corresponding deformed MRI image, producing a registered 3D MRI image for visualizing annotated tissue features from the pre-interventional MRI on the live interventional US image. The disclosed approach leverages multimodal imaging and deep learning models to enhance visualization during interventions by combining superior soft tissue contrast of MRI with real-time US imaging capabilities.

### TECHNICAL FIELD

The present disclosure relates generally to medical imaging and, more specifically, to systems and methods for real-time multimodal deformable image registration for image-guided interventions.

### BACKGROUND

Image-guided interventions are medical procedures that utilize imaging technologies to facilitate the precise targeting of pathological tissues. These interventions commonly employ imaging modalities such as computed tomography (CT), magnetic resonance imaging (MRI), and ultrasound (US) to visualize anatomy and guide the placement of interventional devices. A significant challenge in these procedures is the dynamic nature of internal anatomy, which can undergo motion and deformation due to patient movement, respiratory and cardiac cycles, and interaction with interventional tools.

Ultrasound imaging is frequently selected for its real-time imaging capabilities and the flexibility it offers in accessing various anatomical regions. However, ultrasound imaging has limitations in soft tissue visualization and lesion conspicuity, which limits the usefulness of ultrasound in guiding interventions. While MRI provides superior soft tissue contrast and lesion detection without ionizing radiation, its integration into real-time interventional guidance is complex due to the computational demands of fusing MRI with real-time US images.

CT fluoroscopy guidance is another approach used for abdominal interventions. However, this method is hindered by slow workflows and limited tissue contrast, which is only marginally improved with the use of contrast agents. The use of contrast agents is further limited by patient dosing constraints, reducing their utility in environments where optimized tissue contrast is required promptly. Further, manual processes involved in some CT-US fusion-guided procedures are highly dependent on operator skill and often result in inaccurate alignment due to the use of rigid registration methods.

Despite the superior lesion contrast provided by MRI, especially with the use of contrast agents, leveraging this information for intervention guidance is not straightforward. The computational burden of fusing MRI with real-time US images for guidance during applicator insertion is not conducive to real-time applications. Existing workflows are also largely dependent on manual intervention, with performance varying based on operator expertise. Alternative registration methods, such as analytical deformable registration, introduce significant computation times that are not compatible with the real-time requirements of interventional procedures.

### BRIEF DESCRIPTION

In one embodiment, the present disclosure provides a method for enhancing the visualization of tissue features during an intervention by leveraging the complementary strengths of pre-interventional imaging modalities, such as MRI, CT, or positron emission tomography (PET), and real-time US imaging. The method involves acquiring a pre-interventional three-dimensional (3D) image of an imaging subject in one of the pre-interventional imaging modalities (MRI, CT, or PET). During the intervention, a first interventional 3D US image of the imaging subject is acquired. The pre-interventional 3D image is then registered with the first interventional 3D US image using a trained deformation model specific to the pre-interventional imaging modality and US, such as a trained MR-US deformation model for pre-interventional MRI images. This registration process produces a first deformed 3D image in the pre-interventional imaging modality, aligned with the first interventional 3D US image. As the intervention progresses, a subsequent interventional 3D US image is acquired. To maintain the alignment between the pre-interventional imaging data and the real-time US imaging data, the first interventional 3D US image is registered with the subsequent interventional 3D US image using a trained US-US deformation model. This registration determines a warp field that describes the transformation required to align the first interventional 3D US image with the subsequent interventional 3D US image, accounting for any anatomical motion or deformation that may have occurred between the acquisition of the two US images. The warp field is then applied to the first deformed 3D image in the pre-interventional imaging modality, producing a 3D image in the pre-interventional imaging modality that is registered to the subsequent interventional 3D US image. This registered 3D image enables the visualization of tissue features annotated on the pre-interventional 3D image in real-time on the subsequent interventional 3D US image during the intervention, leveraging the superior soft tissue contrast and lesion conspicuity of the pre-interventional imaging modality while maintaining the real-time imaging capabilities of US. Utilizing the warp field determined between the first interventional 3D US image and the subsequent interventional 3D US image to register the first deformed pre-interventional 3D image to the subsequent US image is computationally more efficient than directly registering the pre-interventional 3D image to the subsequent US image. This approach leverages the deformation field already computed between the two interventional US images acquired at different time points, avoiding the more computationally expensive registration between the pre-interventional imaging modality (e.g., MRI) and the interventional US imaging modality which often exhibit significant differences in appearance and spatial characteristics.

The above advantages and other advantages, and features of the present disclosure will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 is a schematic diagram illustrating an imaging system for acquiring and processing 3D MRI and 3D US images, according to an embodiment of the present disclosure;

FIG. 2 is a block diagram illustrating an image processing device, according to an embodiment of the present disclosure;

FIG. 3 is a schematic diagram illustrating an imaging subject undergoing pre-interventional MRI and US imaging to produce a plurality of 3D MRI images and a plurality of 3D US images which may be used to fine-tune an MR-US deformation model and a US-US deformation model, according to an embodiment of the present disclosure;

FIG. 4 is a flowchart illustrating a method for acquiring pre-interventional 3D MRI and US images, according to an embodiment of the present disclosure;

FIG. 5 is a schematic diagram illustrating an imaging subject undergoing interventional 3D US imaging, showing the use of a trained US-US deformation model to visualize annotated pre-interventional MRI features on the interventional 3D US images, according to an embodiment of the present disclosure;

FIG. 6 is a flowchart illustrating a method for registering a pre-interventional 3D MRI image to an interventional 3D US image using a trained US-US deformation model, according to an embodiment of the present disclosure;

FIG. 7 is a flowchart illustrating a method for acquiring 3D MRI and US images in selected positions and respiratory states, and storing these images in training datasets for MR-US and US-US deformation models, according to an embodiment of the present disclosure;

FIG. 8 is a flowchart illustrating a method for acquiring 3D MRI image images for use in training a MR-US deformation model, according to an embodiment of the present disclosure;

FIG. 9 is a flowchart illustrating a method for acquiring 3D US image images for use in training a MR-US and US-US deformation model, according to an embodiment of the present disclosure;

FIG. 10 is a schematic diagram illustrating a process of training an MR-US deformation model, according to an embodiment of the present disclosure;

FIG. 11 is a flowchart illustrating a method for training an MR-US deformation model, according to an embodiment of the present disclosure;

FIG. 12 is a schematic diagram illustrating a process training a US-US deformation model, according to an embodiment of the present disclosure;

FIG. 13 is a flowchart illustrating a method for training a US-US deformation model, according to an embodiment of the present disclosure;

FIG. 14 shows a pre-interventional MRI image and an interventional US image, with a visualization of the pre-interventional MRI features overlaid on the interventional 3D US image, according to an embodiment of the present disclosure; and

FIG. 15 shows a moving US image, a fixed US image, and a registered image resulting from registering the moving US image to the fixed US image, according to an embodiment of the present disclosure.

### DETAILED DESCRIPTION

The present disclosure provides systems and methods for real-time multimodal deformable image registration tailored for image-guided interventions, particularly addressing the challenges associated with dynamic internal anatomy during medical procedures. Image-guided interventions, which are medical procedures that leverage imaging technologies for precise targeting of pathological tissues, often employ imaging modalities such as computed tomography (CT), magnetic resonance imaging (MRI), and ultrasound (US) to visualize anatomy and guide the placement of interventional devices. A significant challenge in these procedures is the dynamic nature of internal anatomy, which can undergo motion and deformation due to patient movement, respiratory and cardiac cycles, and interaction with interventional tools.

The current disclosure addresses these challenges by providing an improved image registration approach that leverages multimodal imaging and artificial intelligence to enhance the visualization of annotated organ or tumor boundaries during interventions. The approach involves registering pre-interventional three-dimensional (3D) images acquired using modalities such as magnetic resonance imaging (MRI), computed tomography (CT), or positron emission tomography (PET) with real-time three-dimensional (3D) ultrasound (US) images. This enables the visualization of organ or tumor boundaries annotated on the pre-interventional images in real-time on the US images. The approach overcomes the computational limitations of conventional rigid or analytical deformation calculations, which are not conducive to real-time applications due to their extensive computational demands.

The disclosed systems and methods utilize pose-specific, deep-learning based deformation models to fuse pre-interventional imaging data from modalities such as MRI, CT, or PET with real-time 3D US for guiding interventional device placement. In one example, the patients undergo pre-interventional imaging with one of the modalities (MRI, CT, or PET) and US, as well as imaging on the day of the intervention. The pre-interventional imaging captures gated or breath-hold 3D images from the selected modality (MRI, CT, or PET) and multiple 3D US images representing different respiratory states for multiple poses, ranging from supine to decubitus, depending on the anatomy of interest. A deformation model specific to the pre-interventional imaging modality and US, such as an MR-US deformation model for pre-interventional MRI data, is trained to register the pre-interventional images to the US images. Additionally, a US-US deformation model is trained to compute the transformation between pairs of US images for the same patient, capturing the anatomical motion due to respiration and interventional device placement.

The disclosed approach involves acquiring pre-interventional 3D images of the imaging subject using various imaging modalities, such as MRI, CT, or PET, as well as four-dimensional (4D) US images. These pre-interventional images are then utilized to train deformation foundation models. The models are designed to be incrementally trained, with the network model weights initialized from previous training sessions using data from the same imaging subject. This training mechanism offers the advantage of commencing with a single patient's data and iteratively re-training the models with additional patient data, thereby enhancing the robustness of the models in scenarios involving patient re-positioning and internal physiological variations between pre-interventional and interventional imaging sessions. During the intervention, the pre-interventional 3D image, acquired via MRI, CT, or PET, is registered with real-time US images obtained during the intervention, employing the trained deformation models specific to the pre-interventional imaging modality and US. This registration process is performed in substantially real-time, enabling the visualization of lesions or other anatomical features annotated on the pre-interventional images, in conjunction with the real-time US images, to facilitate interventional device placement and procedural confirmation.

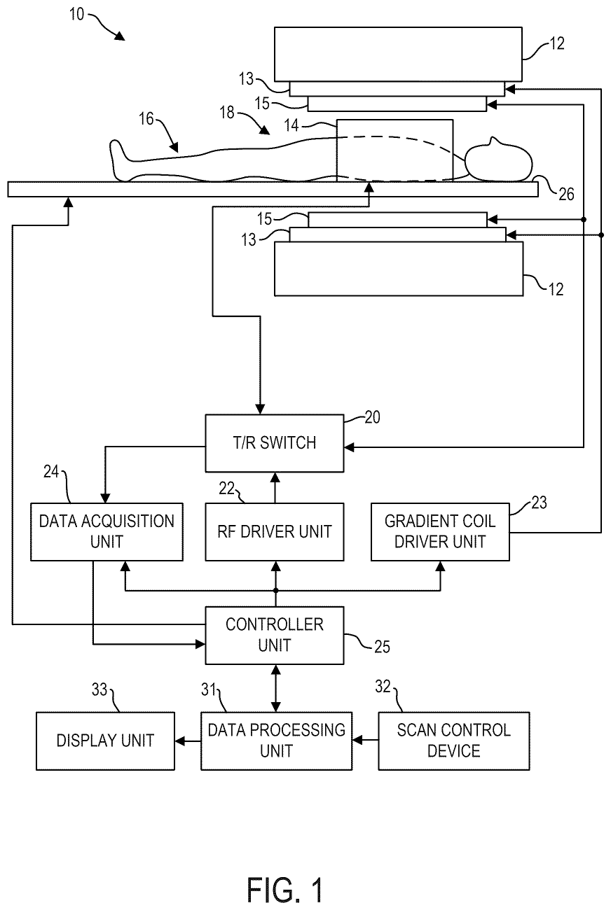

In one embodiment, an MRI apparatus 10, shown in FIG. 1, may acquire high-resolution pre-interventional 3D MRI images of an imaging subject. MRI apparatus 10 is designed to operate in conjunction with a US imaging system to capture pre-interventional US images of the imaging subject in various respiratory states and poses. An image processing system 200 may perform deformation model fine tuning using the acquired pre-interventional 3D MRI images and the pre-interventional 3D US images, and during an intervention the image processing system 200 may employ the fine-tuned deformation models, such as a trained MR-US deformation model 208 and a trained US-US deformation model 210, to align and integrate the pre-interventional 3D MRI image(s) with interventional 3D US images.

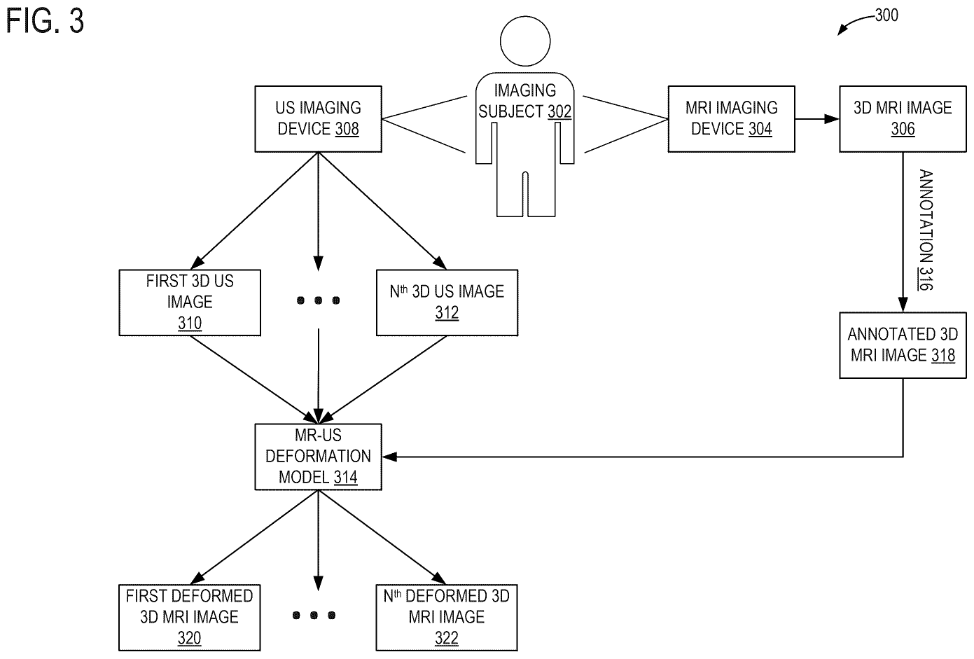

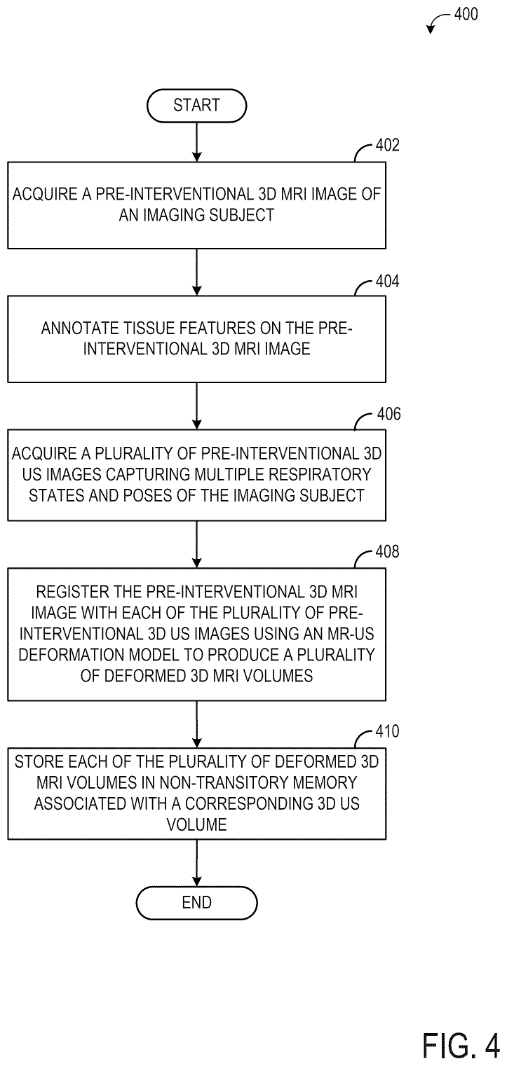

In one example, the image processing system may utilize the process and method shown in FIGS. 3 and 4, respectively, to acquire pre-interventional US and MRI image data from the same imaging subject for fine-tuning the US-US deformation model and the MR-US deformation model. FIG. 3 presents a block diagram of a process 300 for acquiring a high-resolution pre-interventional 3D MRI image and a plurality of pre-interventional 3D US images from the imaging subject (for whom an interventional procedure is planned), capturing multiple respiratory states and poses. These pre-interventional MRI and US images are used to fine-tune the MR-US deformation model 314 for registering the pre-interventional 3D MRI images with the pre-interventional US images, and similarly the US-US deformation model may be fine-tuned by registering the plurality of pre-interventional 3D US images with each other. FIG. 4 is a flowchart of a method 400 for acquiring imaging data for fine-tuning the MR-US deformation model 314 and the US-US deformation model.

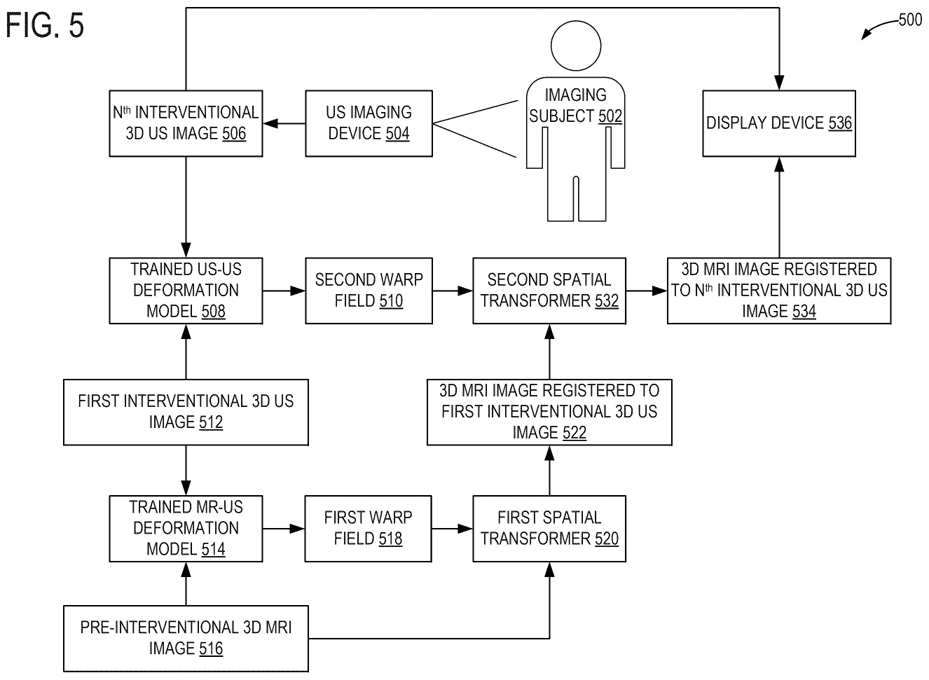

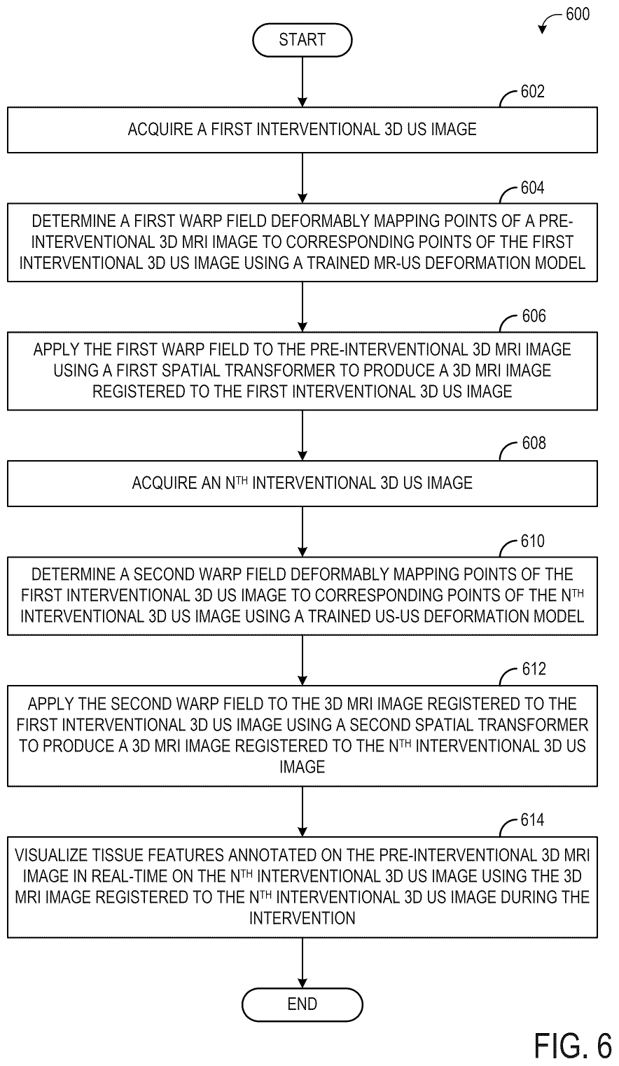

FIGS. 5 and 6 illustrate the registration process during the interventional phase. FIG. 5 is a schematic diagram illustrating an imaging subject 502 undergoing interventional 3D US imaging, showing the use of a trained US-US deformation model 508 to visualize annotated pre-interventional MRI features on the interventional 3D US images. During the intervention, a US imaging device 504 acquires one or more interventional 3D US images, such as a first interventional 3D US image 512 and an N^{th} interventional 3D US image 506, of the imaging subject 502. To leverage the superior soft tissue contrast and lesion conspicuity of pre-interventional MRI data, a pre-interventional 3D MRI image 516 of the imaging subject 502 is registered with the interventional 3D US images using the MR-US deformation model fine-tuned using the data acquired according to FIGS. 3 and 4. The trained US-US deformation model 508 is used to register the first interventional 3D US image 512 with the N^{th} interventional 3D US image 506, determining a second warp field 510 that describes the transformation required to align the two US images. This second warp field 510 is then applied to a 3D MRI image registered to the first interventional 3D US image 522, which was previously produced using the trained MR-US deformation model 514, resulting in a 3D MRI image registered to the N^{th} interventional 3D US image 534. This registered 3D MRI image enables the visualization of tissue features annotated on the pre-interventional 3D MRI image 516 in real-time on the N^{th} interventional 3D US image 506 during the intervention.

FIG. 6 is a flowchart illustrating a method 600 for real-time visualization of tissue features during an intervention using registered MRI and ultrasound images. The method involves acquiring a first interventional 3D US image 602 and determining a first warp field 604 to deformably map points of a pre-interventional 3D MRI image to the first interventional 3D US image using the trained MR-US deformation model. This first warp field is applied to the pre-interventional 3D MRI image to produce a 3D MRI image registered to the first interventional 3D US image 606. As the intervention progresses, an N^{th} interventional 3D US image is acquired 608, and a second warp field is determined 610 to deformably map points of the first interventional 3D US image to the N^{th} interventional 3D US image using the trained US-US deformation model. This second warp field is applied to the 3D MRI image registered to the first interventional 3D US image to produce a 3D MRI image registered to the N^{th} interventional 3D US image 612. Finally, tissue features annotated on the pre-interventional 3D MRI image are visualized in real-time on the N^{th} interventional 3D US image using the registered 3D MRI image 614.

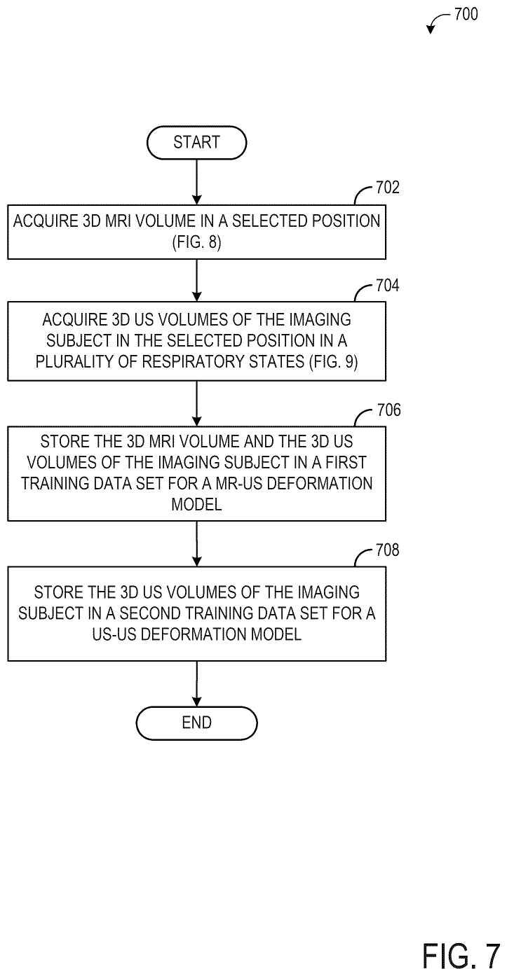

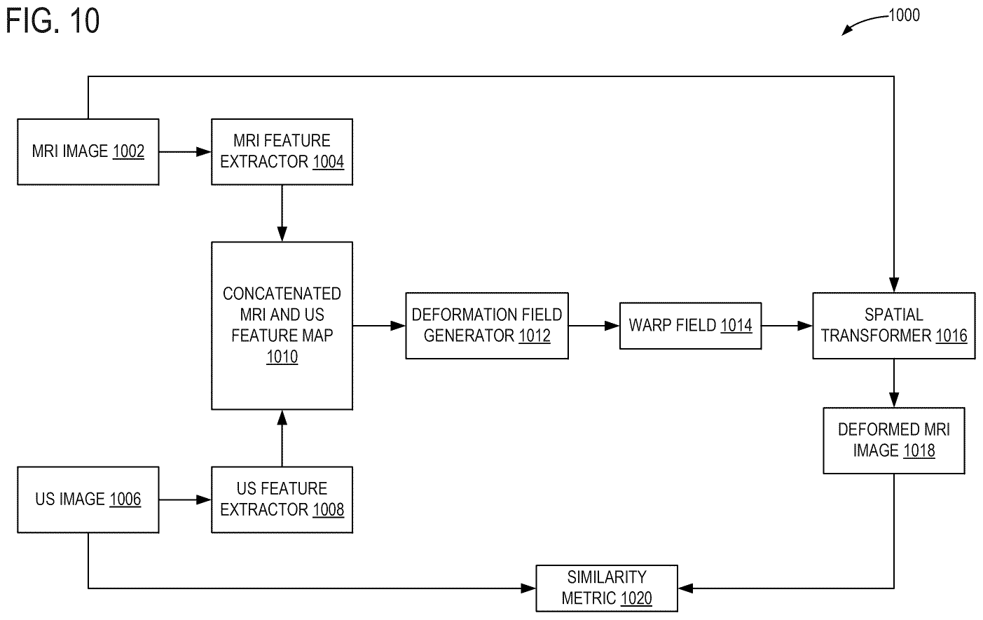

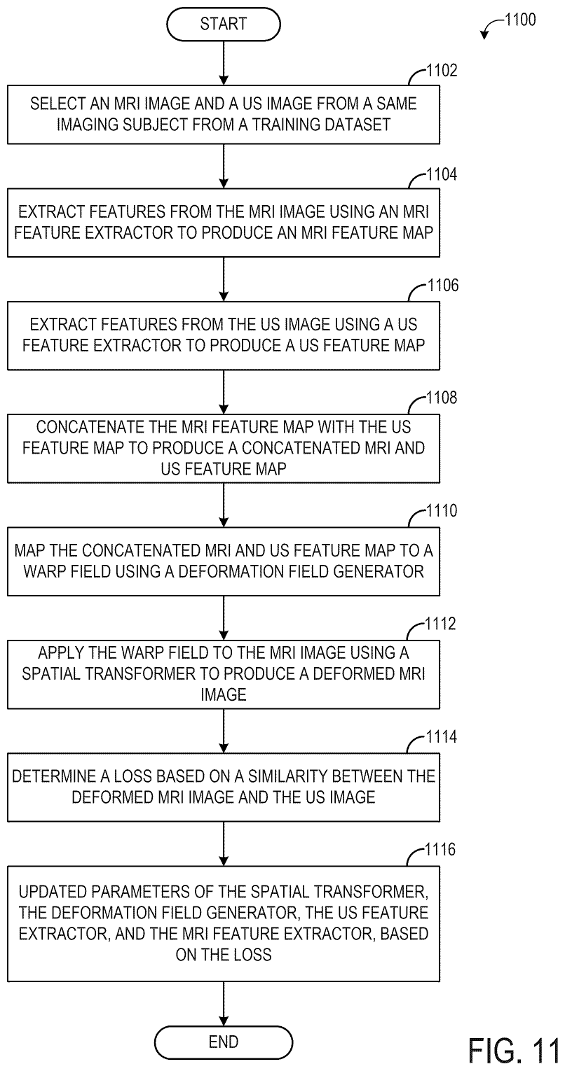

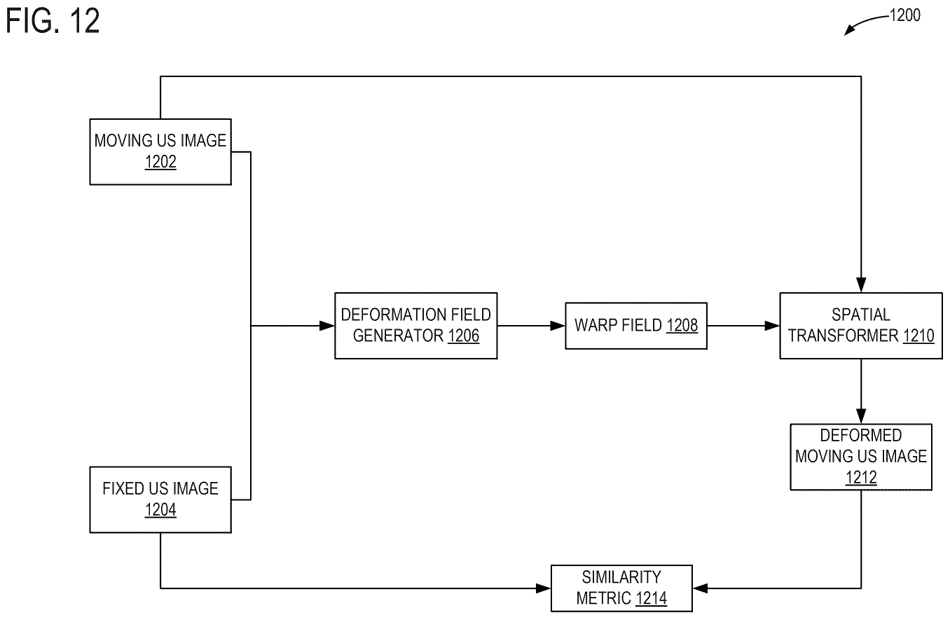

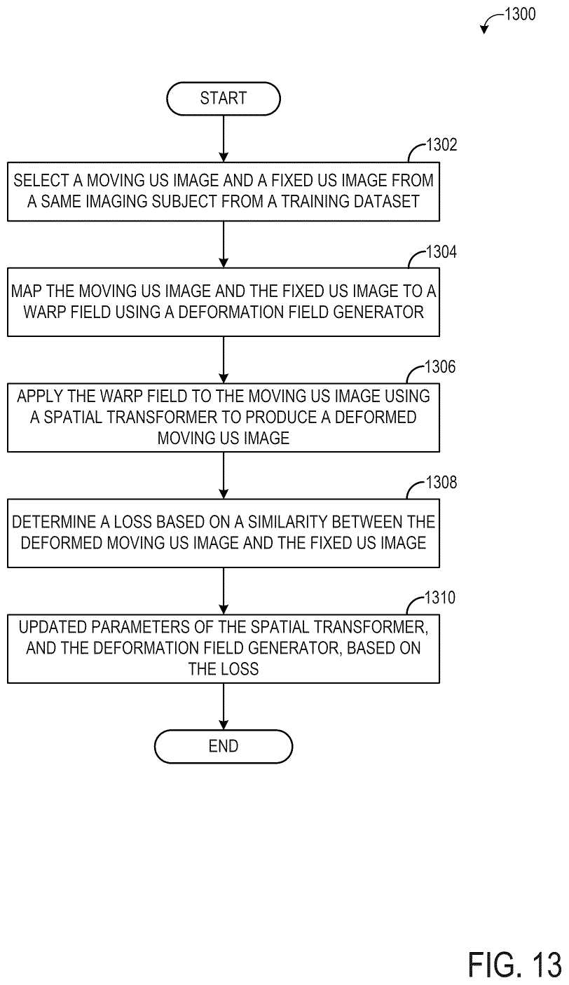

FIGS. 7, 8, and 9 provide flowcharts of methods 700, 800, and 900, respectively, for acquiring MRI and US images in various respiratory states and poses, which are stored in training datasets for the MR-US and US-US deformation models. FIGS. 10 and 11 illustrate the training process for the MR-US deformation model. FIG. 10 is a block diagram of a training process 1000, which encompasses feature extraction and deformation field generation steps. FIG. 11 is a flowchart of a training method 1100, detailing the steps for training the MR-US deformation model using a similarity metric 1020 to update the MR-US deformation model parameters. Similarly, FIGS. 12 and 13 depict the training process for the US-US deformation model. FIG. 12 is a block diagram of a training process 1200, and FIG. 13 is a flowchart of a training method 1300, both focusing on mapping US images to a warp field and adjusting the parameters of the US-US deformation model based on a similarity metric 1214.

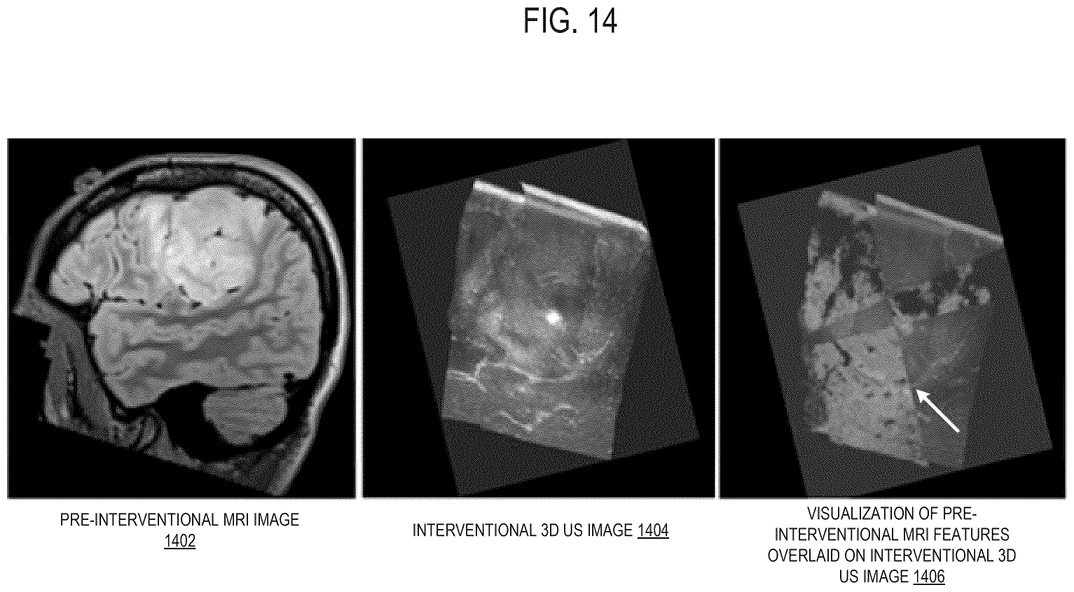

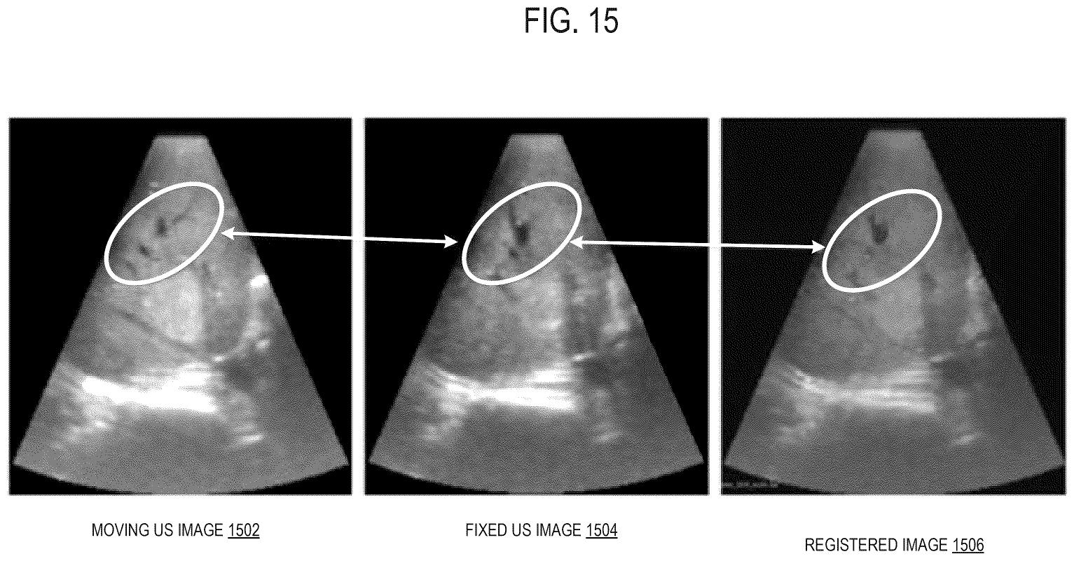

Examples of the efficacy of the currently disclosed approaches for registering high-resolution MRI image images is shown in FIG. 14, which provides a visualization of pre-interventional MRI features overlaid on an interventional 3D US image 1406, while FIG. 15 illustrates the efficacy of the currently disclosed approach for registration of a moving US image 1502 to a fixed US image 1504, resulting in a registered image 1506.

Referring first to FIG. 1, an MRI apparatus 10 is shown, that includes a magnetostatic field magnet unit 12, a gradient coil unit 13, an RF coil unit 14, an RF body coil unit 15 (e.g., image coil unit), a transmit/receive (T/R) switch 20, an RF driver unit 22, a gradient coil driver unit 23, a data acquisition unit 24, a controller unit 25, a patient bed or table 26, a data processing unit 31, a scan control device 32, and a display unit 33. In some embodiments, the RF coil unit 14 is a surface coil, which is a local coil typically placed proximate to the anatomy of interest of a subject 16. Herein, the RF body coil unit 15 is a transmit coil that transmits RF signals, and the local surface of the RF coil unit 14 receives the MR signals. As such, the transmit body coil (e.g., RF body coil unit 15) and the surface receive coil (e.g., RF coil unit 14) are separate but electromagnetically coupled components. The MRI apparatus 10 transmits electromagnetic pulse signals to the subject 16 placed in an imaging space 18 with a static magnetic field formed to perform a scan for obtaining magnetic resonance signals from the subject 16. One or more images of the subject 16 can be reconstructed based on the magnetic resonance signals thus obtained by the scan.

The magnetostatic field magnet unit 12 includes, for example, an annular superconducting magnet, which is mounted within a toroidal vacuum vessel. The magnet defines a cylindrical space surrounding the subject 16 and generates a constant primary magnetostatic field B₀.

The MRI apparatus 10 also includes a gradient coil unit 13 that forms a gradient magnetic field in the imaging space 18 so as to provide the magnetic resonance signals received by the RF coil arrays with three-dimensional positional information. The gradient coil unit 13 includes three gradient coil systems, each of which generates a gradient magnetic field along one of three spatial axes perpendicular to each other, and generates a gradient field in each of a frequency encoding direction, a phase encoding direction, and a slice selection direction in accordance with the imaging condition. More specifically, the gradient coil unit 13 applies a gradient field in the slice selection direction (or scan direction) of the subject 16, to select the slice; and the RF body coil unit 15 or the local RF coil arrays may transmit an RF pulse to a selected slice of the subject 16. The gradient coil unit 13 also applies a gradient field in the phase encoding direction of the subject 16 to phase encode the magnetic resonance signals from the slice excited by the RF pulse. The gradient coil unit 13 then applies a gradient field in the frequency encoding direction of the subject 16 to frequency encode the magnetic resonance signals from the slice excited by the RF pulse.

The RF coil unit 14 is disposed, for example, to enclose the region to be imaged of the subject 16. In some examples, the RF coil unit 14 may be referred to as the surface coil or the receive coil. In the static magnetic field space or imaging space 18 where a static magnetic field B₀ is formed by the magnetostatic field magnet unit 12, the RF body coil unit 15 transmits, based on a control signal from the controller unit 25, an RF pulse that is an electromagnet wave to the subject 16 and thereby generates a high-frequency magnetic field B₁. This excites a spin of protons in the slice to be imaged of the subject 16. The RF coil unit 14 receives, as a magnetic resonance signal, the electromagnetic wave generated when the proton spin thus excited in the slice to be imaged of the subject 16 returns into alignment with the initial magnetization vector. In some embodiments, the RF coil unit 14 may transmit the RF pulse and receive the MR signal. In other embodiments, the RF coil unit 14 may only be used for receiving the MR signals, but not transmitting the RF pulse.

The RF body coil unit 15 is disposed, for example, to enclose the imaging space 18, and produces RF magnetic field pulses orthogonal to the main magnetic field B₀ produced by the magnetostatic field magnet unit 12 within the imaging space 18 to excite the nuclei. In contrast to the RF coil unit 14, which may be disconnected from the MRI apparatus 10 and replaced with another RF coil unit, the RF body coil unit 15 is fixedly attached and connected to the MRI apparatus 10. Furthermore, whereas local coils such as the RF coil unit 14 can transmit to or receive signals from only a localized region of the subject 16, the RF body coil unit 15 generally has a larger coverage area. The RF body coil unit 15 may be used to transmit or receive signals to the whole body of the subject 16, for example. Using receive-only local coils and transmit body coils provides a uniform RF excitation and good image uniformity at the expense of high RF power deposited in the subject. For a transmit-receive local coil, the local coil provides the RF excitation to the region of interest and receives the MR signal, thereby decreasing the RF power deposited in the subject. It should be appreciated that the particular use of the RF coil unit 14 and/or the RF body coil unit 15 depends on the imaging application.

The T/R switch 20 can selectively electrically connect the RF body coil unit 15 to the data acquisition unit 24 when operating in receive mode, and to the RF driver unit 22 when operating in transmit mode. Similarly, the T/R switch 20 can selectively electrically connect the RF coil unit 14 to the data acquisition unit 24 when the RF coil unit 14 operates in receive mode, and to the RF driver unit 22 when operating in transmit mode. When the RF coil unit 14 and the RF body coil unit 15 are both used in a single scan, for example if the RF coil unit 14 is configured to receive MR signals and the RF body coil unit 15 is configured to transmit RF signals, then the T/R switch 20 may direct control signals from the RF driver unit 22 to the RF body coil unit 15 while directing received MR signals from the RF coil unit 14 to the data acquisition unit 24. The coils of the RF body coil unit 15 may be configured to operate in a transmit-only mode or a transmit-receive mode. The coils of the RF coil unit 14 may be configured to operate in a transmit-receive mode or a receive-only mode.

The RF driver unit 22 includes a gate modulator (not shown), an RF power amplifier (not shown), and an RF oscillator (not shown) that are used to drive the RF coils (e.g., RF body coil unit 15) and form a high-frequency magnetic field in the imaging space 18. The RF driver unit 22 modulates, based on a control signal from the controller unit 25 and using the gate modulator, the RF signal received from the RF oscillator into a signal of predetermined timing having a predetermined envelope. The RF signal modulated by the gate modulator is amplified by the RF power amplifier and then output to the RF body coil unit 15.

The gradient coil driver unit 23 drives the gradient coil unit 13 based on a control signal from the controller unit 25 and thereby generates a gradient magnetic field in the imaging space 18. The gradient coil driver unit 23 includes three systems of driver circuits (not shown) corresponding to the three gradient coil systems included in the gradient coil unit 13.

The data acquisition unit 24 includes a pre-amplifier (not shown), a phase detector (not shown), and an analog/digital converter (not shown) used to acquire the magnetic resonance signals received by the RF coil unit 14. In the data acquisition unit 24, the phase detector phase detects, using the output from the RF oscillator of the RF driver unit 22 as a reference signal, the magnetic resonance signals received from the RF coil unit 14 and amplified by the pre-amplifier, and outputs the phase-detected analog magnetic resonance signals to the analog/digital converter for conversion into digital signals. The digital signals thus obtained are output to the data processing unit 31.

The MRI apparatus 10 includes a table 26 for placing the subject 16 thereon. The subject 16 may be moved inside and outside the imaging space 18 by moving the table 26 based on control signals from the controller unit 25.

The controller unit 25 includes a computer and a non-transitory computer-readable storage medium on which computer-executable instructions are stored. When executed by the computer, the instructions cause various components of the apparatus 10 to carry out operations corresponding to predetermined scanning protocols. The non-transitory computer-readable storage medium may comprise one or more of a solid-state drive (SSD), a hard disk drive (HDD), a hybrid drive, an optical disc (e.g., CD, DVD, Blu-ray), a flash memory device, a random access memory (RAM) device, a read-only memory (ROM) device, or any other suitable non-transitory storage medium. In some embodiments, the non-transitory computer-readable storage medium may be a cloud-based or network-attached storage system accessible by the controller unit 25 over a wired or wireless network connection.

The controller unit 25 is connected to the scan control device 32 and processes the operation signals input to the scan control device 32 and furthermore controls the table 26, RF driver unit 22, gradient coil driver unit 23, and data acquisition unit 24 by outputting control signals to them. The controller unit 25 also controls, to obtain a desired image, the data processing unit 31 and the display unit 33 based on operation signals received from the scan control device 32.

The scan control device 32 includes user input devices such as a touchscreen, keyboard and a mouse. The scan control device 32 is used by an operator, for example, to input such data as an imaging protocol and to set a region where an imaging sequence is to be executed. The data about the imaging protocol and the imaging sequence execution region are output to the controller unit 25.

The data processing unit 31 includes a computer and a recording medium on which a program to be executed by the computer to perform predetermined data processing is recorded. The data processing unit 31 is connected to the controller unit 25 and performs data processing based on control signals received from the controller unit 25. The data processing unit 31 is also connected to the data acquisition unit 24 and generates spectrum data by applying various image processing operations to the magnetic resonance signals output from the data acquisition unit 24.

The display unit 33 includes a display device and displays an image on the display screen of the display device based on control signals received from the controller unit 25. The display unit 33 displays, for example, an image regarding an input item about which the operator inputs operation data from the scan control device 32. The display unit 33 also displays a two-dimensional (2D) slice image or three-dimensional (3D) image of the subject 16 generated by the data processing unit 31.

During an MRI scan using the MRI apparatus 10, a subject may be positioned within the imaging space 18 and an acquisition protocol may be carried out to obtain MR signals of the subject. The acquisition protocol may include a plurality of pulse sequences where in each pulse sequence, contrast is prepared via one or more RF pulses applied by the RF body coil unit 15 and the gradient coil unit 13 is controlled to spatially encode the resultant MR signals. The spatially-encoded MR signals are received by the RF coil unit 14 are digitized and stored in k-space. Thus, k-space data or a k-space dataset may refer to the raw MR signals prior to processing into an image. In some examples, one line of k-space may be filled with the raw MR signals per pulse sequence (also referred to as repetition time). In other examples, one line of k-space may be filled with the raw MR signals per echo, where more than one echo is generated per pulse sequence/repetition time. The k-space data may also be referred to as imaging data or MR data herein.

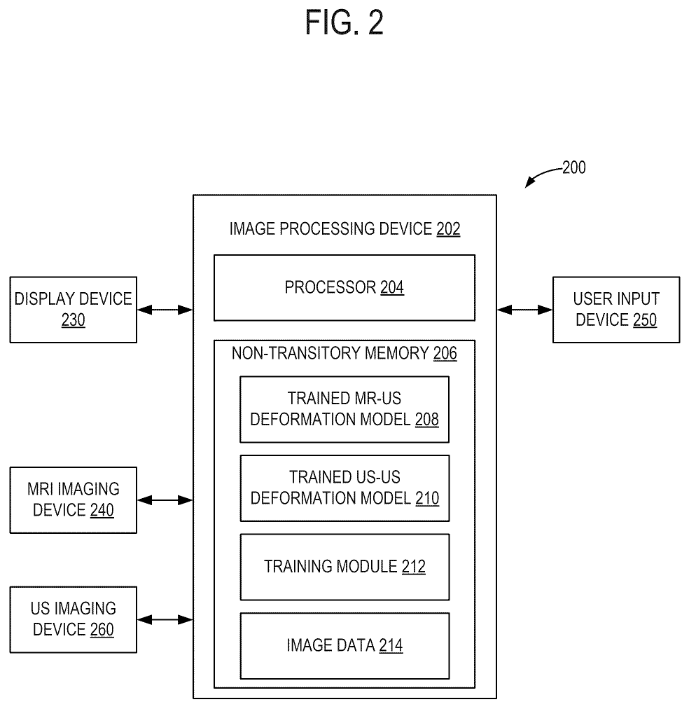

Referring to FIG. 2, an image processing system 200 for deformable registration of multi-modal medical images is disclosed, in accordance with an exemplary embodiment. The system is configured to facilitate registration of MRI and US images during image-guided interventions. This system utilizes computational models and algorithms to enhance the precision and efficiency of medical procedures by providing visualization of tissue features and anatomical structures.

The image processing system 200 includes an image processing device 202, a user input device 250, a display device 230, an MRI imaging device 240, and a US imaging device 260. Each component is configured to operate in concert to deliver imaging capabilities for successful image-guided interventions.

The image processing device 202 comprises a processor 204, a non-transitory memory 206, and various modules and data stored within the memory 206. The processor 204 is configured to execute machine-readable instructions that control the operation of the image processing system 200. The processor 204 may include individual components that are distributed throughout two or more devices, which may be remotely located and/or configured for coordinated processing.

Non-transitory memory 206 stores components such as a trained MR-US deformation model 208, a trained US-US deformation model 210, a training module 212, and image data 214. The trained MR-US deformation model 208 and the trained US-US deformation model 210 are deep learning models trained to perform deformable registration of MRI and US images. These models handle variations and movements of internal anatomy during interventions, providing accuracy in image registration.

The training module 212 is configured for training and fine-tuning the deformation models using new data as it becomes available. This module can utilize network model weights initialized from previous training sessions, allowing for incremental learning and adaptation to new patient data or different anatomical features. The image data 214 includes pre-interventional and interventional MRI and US images, which are used for training the models and for guidance during medical procedures.

The user input device 250, such as a keyboard, mouse, or touchscreen, enables medical professionals to interact with the image processing system 200. It allows users to input commands, adjust settings, and manipulate image data during procedures.

The display device 230 is configured to present the registered images and other relevant information to the medical professionals during an intervention. It may show overlays of MRI features on US images, enhancing the visibility of critical tissue features and anatomical structures. The display device 230 includes features such as high resolution and real-time response capabilities to ensure clarity and immediacy of visual information.

The MRI imaging device 240 and the US imaging device 260 are configured to acquire the medical images used by the image processing system 200. The MRI imaging device 240 captures high-resolution 3D images of the patient's anatomy, providing detailed information enabling accurate registration and intervention planning. The US imaging device 260 offers real-time imaging capabilities, allowing for substantially real time visualization of the anatomy and the effects of the intervention.

In some embodiments, the MRI imaging device 240 and the US imaging device 260 may be integrated into a simultaneous MR and ultrasound imaging system with a 3D US probe. This integration facilitates the acquisition of aligned MRI and US images, reducing the complexity of subsequent image registration processes.

In alternative embodiments, the image processing system 200 may include additional components such as a network interface for connecting to hospital information systems, facilitating integration of image data with patient records and other medical data. Additionally, the system may support remote access capabilities, enabling specialists to participate in procedures or review images from distant locations.

Referring to FIG. 3, a process 300 is shown for acquiring and preparing training data for fine-tuning an MR-US deformation model 314 for registering pre-interventional MRI images with ultrasound images. The MRI imaging device 304 is used to acquire a pre-interventional 3D MRI image 306 of the imaging subject 302. The 3D MRI image 306 provides high-resolution anatomical details of the region of interest within the imaging subject 302, offering superior soft tissue contrast and lesion conspicuity compared to other imaging modalities.

In one embodiment, the 3D MRI image 306 is acquired using a gated or breath-hold imaging technique to minimize artifacts caused by respiratory motion or other patient movement during the image acquisition process. In another embodiment, the 3D MRI image 306 is acquired with the administration of a contrast agent to further enhance the visualization of specific tissues or anatomical structures of interest.

Once the 3D MRI image 306 is acquired, tissue features of interest, such as organ boundaries, tumor boundaries, or other relevant anatomical structures, are demarcated as indicated by annotation 316 on the 3D MRI image 306, resulting in an annotated 3D MRI image 318. In one embodiment, the tissue features are manually annotated by a radiologist or other medical professional using specialized software tools. The annotation process may involve segmenting or delineating the boundaries of the tissue features on individual 2D slices of the 3D MRI image 306, or using semi-automated or automated segmentation algorithms. In an alternative embodiment, the tissue features are automatically annotated using machine learning or deep learning techniques trained on labeled datasets of MRI images. These techniques may involve convolutional neural networks or other deep learning architectures capable of accurately detecting and segmenting various tissue types and anatomical structures within the 3D MRI image 306.

In parallel with the acquisition and annotation of the 3D MRI image 306, the US imaging device 308 is used to acquire a plurality of pre-interventional 3D US images 310, 312, capturing multiple respiratory states and poses of the imaging subject 302. These pre-interventional 3D US images 310, 312 may be obtained using a 3D ultrasound probe. In one embodiment, the plurality of pre-interventional 3D US images 310, 312 are acquired at a predetermined imaging frequency (e.g., 3-4 images per second) over a predetermined duration of time, capturing the anatomical motion and deformation of the region of interest due to respiration and changes in patient positioning. In another embodiment, the plurality of pre-interventional 3D US images 310, 312 are acquired while the imaging subject 302 is instructed to hold their breath at different respiratory states (e.g., full inspiration, full expiration, and intermediate states). Additionally, the plurality of pre-interventional 3D US images 310, 312 may be acquired with the imaging subject 302 in different poses or positions, such as supine, decubitus, or other orientations relevant to the planned interventional procedure and the anatomical region of interest.

The annotated 3D MRI image 318 and the plurality of pre-interventional 3D US images 310, 312 are used to fine-tune the MR-US deformation model 314 for the imaging subject 302. The MR-US deformation model 314 is a deep learning-based model that is trained to accurately align and register MRI and ultrasound image data, accounting for the inherent differences in image appearance and characteristics between these two imaging modalities. In one embodiment, the MR-US deformation model 314 is based on a CNN architecture that takes the annotated 3D MRI image 318 and a pre-interventional 3D US image (e.g., 310) as input during training, and learns to output a deformation field that maps the annotated 3D MRI image 318 to the corresponding pre-interventional 3D US image 310. This deformation field is then applied to the annotated 3D MRI image 318 using a spatial transformer, producing a first deformed 3D MRI image 320 that aligns with the pre-interventional 3D US image 310. In one embodiment, the MR-US deformation model 314 is an unsupervised deep learning model that does not require ground truth deformation fields for training. Instead, the model is trained using a similarity metric, such as normalized cross-correlation or mutual information, to maximize the alignment between the deformed 3D MRI image 320 and the corresponding pre-interventional 3D US image 310. This unsupervised approach allows the model to learn the complex, non-linear deformations required to register MRI and ultrasound data without the need for manually annotated ground truth deformation fields.

The MR-US deformation model 314 is trained on each of the pre-interventional 3D US images 310, 312, resulting in a plurality of deformed 3D MRI images 320, 322, each corresponding to a specific respiratory state or pose of the imaging subject 302 captured by the respective pre-interventional 3D US image 310, 312.

Referring to FIG. 4, a flowchart of a method 400 for fine-tuning a trained MR-US deformation model using pre-interventional 3D MRI images and pre-interventional 3D US images of the same imaging subject is shown. The method 400 outlines the steps involved in acquiring and processing the necessary imaging data to fine-tune the MR-US deformation model, which is a deep learning-based model designed to accurately align and register MRI and US data.

The method 400 begins at operation 402, where a pre-interventional three-dimensional (3D) MRI image of an imaging subject is acquired. In one embodiment, the pre-interventional 3D MRI image is obtained using an MRI apparatus, such as the MRI apparatus 10 shown in FIG. 1. The MRI apparatus 10 may employ various imaging techniques, including gated or breath-hold imaging, to capture a high-resolution 3D image of the imaging subject's anatomy while minimizing artifacts caused by respiratory motion or other patient movements.

At operation 404, tissue features of interest are annotated on the pre-interventional 3D MRI image acquired in operation 402. In one embodiment, the tissue features are manually annotated by a radiologist or other medical professional using specialized software tools. The annotation process may involve segmenting or delineating the boundaries of the tissue features on individual two-dimensional (2D) slices of the 3D MRI image, or using semi-automated or automated segmentation algorithms. The tissue features annotated may include organ boundaries, tumor boundaries, or other relevant anatomical structures of interest for the planned interventional procedure. In an alternative embodiment, the tissue features are automatically annotated using machine learning or deep learning techniques trained on labeled datasets of MRI images. These techniques may involve convolutional neural networks or other deep learning architectures capable of accurately detecting and segmenting various tissue types and anatomical structures within the 3D MRI image.

At operation 406, a plurality of pre-interventional 3D US images of the same imaging subject are acquired, capturing multiple respiratory states and poses. In one embodiment, the plurality of pre-interventional 3D US images are obtained using a 3D ultrasound probe or transducer. The pre-interventional 3D US images may be acquired at a predetermined imaging frequency over a predetermined duration of time, capturing the anatomical motion and deformation of the region of interest due to respiration and changes in patient positioning. In another embodiment, the plurality of pre-interventional 3D US images are acquired while the imaging subject is instructed to hold their breath at different respiratory states, such as full inspiration, full expiration, and intermediate states. Additionally, the plurality of pre-interventional 3D US images may be acquired with the imaging subject in different poses or positions, such as supine, decubitus, or other orientations relevant to the planned interventional procedure and the anatomical region of interest.

At operation 408, the pre-interventional 3D MRI image acquired in operation 402 is registered with each of the plurality of pre-interventional 3D US images acquired in operation 406 using an MR-US deformation model. The MR-US deformation model is a deep learning-based model that is trained to accurately align and register MRI and ultrasound image data, accounting for the inherent differences in image appearance and characteristics between these two imaging modalities. In one embodiment, the MR-US deformation model is based on a convolutional neural network (CNN) architecture that takes the pre-interventional 3D MRI image and a pre-interventional 3D US image as input during training. The model learns to output a deformation field that maps the pre-interventional 3D MRI image to the corresponding pre-interventional 3D US image. This deformation field is then applied to the pre-interventional 3D MRI image using a spatial transformer, producing a deformed 3D MRI image that aligns with the pre-interventional 3D US image. The registration process performed at operation 408 results in a plurality of deformed 3D MRI images, each corresponding to a specific pre-interventional 3D US image and representing the pre-interventional 3D MRI image deformed to align with that particular US image.

At operation 410, each of the plurality of deformed 3D MRI images produced in operation 408 is stored in non-transitory memory, such as a hard disk drive or solid-state drive, associated with the corresponding pre-interventional 3D US image. In one embodiment, the deformed 3D MRI images and their associated pre-interventional 3D US images are stored in a structured database or file system, where each MRI image is linked to the corresponding set of US images acquired in the same respiratory state and pose. This organization facilitates efficient retrieval and processing of the imaging data during the interventional phase, when the deformed 3D MRI images will be used in conjunction with interventional US images for real-time visualization of tissue features. In an alternative embodiment, the deformed 3D MRI images and their associated pre-interventional 3D US images are stored along with metadata describing the imaging parameters, patient information, respiratory state annotations, and other relevant details. This metadata can be used for data management, quality control, and potential future refinements or extensions of the MR-US deformation model.

Following operation 410, the method 400 ends, having acquired and processed the pre-interventional imaging data for fine-tuning the MR-US deformation model. The fine-tuned MR-US deformation model, along with the stored deformed 3D MRI images and their associated pre-interventional 3D US images, will be used during the interventional phase to enable real-time visualization of tissue features annotated on the pre-interventional MRI data on live interventional US images, enhancing the guidance and precision of the interventional procedure.

Referring to FIG. 5, an interventional image registration process 500 for registering pre-interventional MRI images to interventional 3D ultrasound images is shown. This process enables the visualization of tissue features annotated on pre-interventional MRI images in real-time on interventional 3D US images, enhancing the guidance and accuracy of interventional procedures.

The process 500 begins with an imaging subject 502 undergoing an intervention. During the intervention, a US imaging device 504 acquires one or more interventional 3D US images, such as a first interventional 3D US image 512 and an N^{th} interventional 3D US image 506, of the imaging subject 502. These interventional 3D US images capture the anatomical region of interest in real-time, providing up-to-date information about the subject's anatomy and the position of any interventional devices or instruments.

To leverage the superior soft tissue contrast and lesion conspicuity of pre-interventional MRI data, a pre-interventional 3D MRI image 516 of the imaging subject 502 is registered with the interventional 3D US images using a two-step process involving a trained MR-US deformation model 514 and a trained US-US deformation model 508.

In the first step, the pre-interventional 3D MRI image 516 is registered with the first interventional 3D US image 512 using the trained MR-US deformation model 514. The trained MR-US deformation model 514 is a deep learning-based model that utilizes features derived from the pre-interventional 3D MRI image 516 and the first interventional 3D US image 512, including radiomics features extracted from the images and Gaussian Mixture Model tissue class probabilities calculated for each voxel in the images. The radiomics features may include descriptors of intensity, shape, texture, and other quantitative characteristics within the images. The Gaussian Mixture Model tissue class probabilities provide a voxel-wise estimation of the probability that each voxel belongs to different tissue classes such as fat, muscle, organ tissues, etc. By leveraging these derived features, the trained MR-US deformation model 514 can account for the inherent differences in image appearance and characteristics between MRI and ultrasound data. In one embodiment, the trained MR-US deformation model 514 is based on a convolutional neural network (CNN) architecture that takes the pre-interventional 3D MRI image 516, the first interventional 3D US image 512, and the derived radiomics and tissue probability features as input, and outputs a first warp field 518 that encodes the transformation required to align the pre-interventional 3D MRI image 516 with the first interventional 3D US image 512.

The first warp field 518 is then applied to the pre-interventional 3D MRI image 516 using a first spatial transformer 520. The first spatial transformer 520 is a differentiable module that applies the first warp field 518 to the pre-interventional 3D MRI image 516 using a sampling kernel, such as bilinear or trilinear interpolation, to produce a 3D MRI image registered to the first interventional 3D US image 522.

In the second step, the 3D MRI image registered to the first interventional 3D US image 522 is further registered with the N^{th} interventional 3D US image 506 using the trained US-US deformation model 508. Similar to the MR-US deformation model, the trained US-US deformation model 508 utilizes radiomics features and Gaussian Mixture Model tissue class probabilities derived from the first interventional 3D US image 512 and the N^{th} interventional 3D US image 506 to account for differences in appearance between the two US images caused by factors such as anatomical motion and deformation. The trained US-US deformation model 508 is based on a CNN architecture that takes the first interventional 3D US image 512, the N^{th} interventional 3D US image 506, and the derived radiomics and tissue probability features as input, and outputs a second warp field 510 that describes the transformation required to align the first interventional 3D US image 512 with the N^{th} interventional 3D US image 506.

The second warp field 510 is then applied to the 3D MRI image registered to the first interventional 3D US image 522 using a second spatial transformer 532. The second spatial transformer 532 is a differentiable module that applies the second warp field 510 to the 3D MRI image registered to the first interventional 3D US image 522 using a sampling kernel to produce a 3D MRI image registered to the N^{th} interventional 3D US image 534.

In an alternative embodiment, the second spatial transformer 532 applies the second warp field 510 to the 3D MRI image registered to the first interventional 3D US image 522 using a non-rigid registration algorithm, such as B-spline or free-form deformation, to produce the 3D MRI image registered to the N^{th} interventional 3D US image 534.

The 3D MRI image registered to the N^{th} interventional 3D US image 534 can then be displayed on a display device 536, along with the N^{th} interventional 3D US image 506. By overlaying the registered 3D MRI image onto the N^{th} interventional 3D US image 506, tissue features annotated on the pre-interventional MRI image can be visualized in real-time on the N^{th} interventional 3D US image 506, providing valuable guidance for the interventional procedure.

In one embodiment, the display device 536 is a high-resolution monitor or display panel capable of displaying both the N^{th} interventional 3D US image 506 and the 3D MRI image registered to the N^{th} interventional 3D US image 534 simultaneously. The display device 536 may support various visualization modes, such as side-by-side or overlaid views, to facilitate the comparison and integration of the two image modalities.

In another embodiment, the process 500 may incorporate additional pre-interventional imaging modalities or data sources, such as computed tomography (CT) or positron emission tomography (PET), by training additional deformation models and integrating them into the registration pipeline. This flexibility allows the system to leverage the strengths of various imaging modalities and provide a comprehensive multimodal visualization for interventional guidance.

The interventional image registration process 500 overcomes the computational limitations of conventional rigid or analytical deformation calculations, which are not conducive to real-time applications due to their extensive computational demands. By leveraging the trained US-US deformation model 508 and the pre-computed 3D MRI image registered to the first interventional 3D US image 522, the process 500 enables real-time visualization of tissue features during interventions, enhancing the accuracy and efficiency of the procedures.

Referring to FIG. 6, a flowchart of a method 600 for real-time visualization of tissue features during an intervention using registered MRI and ultrasound images is shown. The method 600 enables the integration of high-resolution anatomical information from pre-interventional MRI data with real-time ultrasound imaging data, allowing for enhanced visualization of tissue features during interventional procedures.

The method 600 begins at operation 602, where a first interventional 3D US image is acquired during an intervention. In one embodiment, the interventional 3D US image is captured using a 3D US probe or transducer positioned near the anatomical region of interest during the intervention. The 3D US probe may be capable of acquiring real-time 3D US images, enabling the visualization of the anatomical region and any interventional devices or instruments in real-time. The acquisition of the interventional 3D US image may be triggered manually by an operator or automatically based on predefined criteria, such as the detection of an interventional device within the imaging field of view.

At operation 604, a first warp field deformably mapping points of a pre-interventional 3D MRI image to corresponding points of the interventional 3D US image acquired in operation 602 is determined using a trained MR-US deformation model. In one embodiment, the trained MR-US deformation model is a deep learning-based model that has been trained to accurately align and register MRI and ultrasound image data, accounting for the inherent differences in image appearance and characteristics between these two imaging modalities. The trained MR-US deformation model may take the pre-interventional 3D MRI image and the interventional 3D US image as inputs and output the first warp field, which represents the deformation or displacement field required to align the pre-interventional 3D MRI image with the interventional 3D US image. The first warp field may be a dense warp field, where each voxel in the pre-interventional 3D MRI image is associated with a displacement vector, or a sparse warp field, where the displacements are defined only at a subset of control points, and the displacements for the remaining voxels are interpolated from the control point displacements.

At operation 606, the first warp field determined in operation 604 is applied to the pre-interventional 3D MRI image using a first spatial transformer to produce a 3D MRI image registered to the first interventional 3D US image acquired in operation 602. In one embodiment, the first spatial transformer is a differentiable module that applies the first warp field to the pre-interventional 3D MRI image using a sampling kernel, such as bilinear or trilinear interpolation. This operation effectively warps or deforms the pre-interventional 3D MRI image to align it with the interventional 3D US image, accounting for any anatomical motion or deformation that may have occurred between the acquisition of the two images.

The method 600 then proceeds to operation 608, where an N^{th} interventional 3D US image is acquired during the intervention. This operation may be repeated multiple times throughout the intervention, capturing the anatomical region of interest at different time points or in different states of deformation due to factors such as patient movement, respiratory motion, or interactions with interventional devices.

At operation 610, a second warp field deformably mapping points of the first interventional 3D US image acquired in operation 602 to corresponding points of the N^{th} interventional 3D US image acquired in operation 608 is determined using a trained US-US deformation model. The trained US-US deformation model is a deep learning-based model that has been trained to compute the transformation between pairs of US images representing different respiratory states or poses for the same patient. In one embodiment, the trained US-US deformation model takes the first interventional 3D US image acquired in operation 602 and the N^{th} interventional 3D US image acquired in operation 608 as inputs and outputs the second warp field. The second warp field represents the deformation or displacement field required to align the first interventional 3D US image acquired in operation 602 with the N^{th} interventional 3D US image acquired in operation 608, accounting for any anatomical motion or deformation that may have occurred between the acquisition of the two US images.

At operation 612, the second warp field determined in operation 610 is applied to the 3D MRI image registered to the first interventional 3D US image acquired in operation 602 using a second spatial transformer to produce a 3D MRI image registered to the N^{th} interventional 3D US image acquired in operation 608. In one embodiment, the second spatial transformer is a differentiable module that applies the second warp field to the 3D MRI image registered to the first interventional 3D US image acquired in operation 602 using a sampling kernel, such as bilinear or trilinear interpolation. Operation 612 effectively warps or deforms the 3D MRI image registered to the first interventional 3D US image acquired in operation 602 to align it with the N^{th} interventional 3D US image acquired in operation 608. By applying the second warp field, the resulting 3D MRI image is registered to the most recent interventional 3D US image, accounting for any anatomical motion or deformation that may have occurred between the acquisition of the first interventional 3D US image acquired in operation 602 and the N^{th} interventional 3D US image acquired in operation 608.

At operation 614, tissue features annotated on the pre-interventional 3D MRI image are visualized in real-time on the N^{th} interventional 3D US image acquired in operation 608 using the 3D MRI image registered to the N^{th} interventional 3D US image produced in operation 612. In one embodiment, the tissue features annotated on the pre-interventional 3D MRI image, such as organ or tumor boundaries, are overlaid onto the N^{th} interventional 3D US image using the 3D MRI image registered to the N^{th} interventional 3D US image. This overlay process may involve techniques such as alpha blending, where the intensities of the MRI and US images are combined using a weighted sum, allowing the tissue features annotated on the pre-interventional MRI image to be visible on the interventional US image while preserving the real-time imaging capabilities of the US modality. Alternatively, the tissue features annotated on the pre-interventional 3D MRI image may be segmented and rendered as 3D surfaces or meshes, which can then be superimposed onto the N^{th} interventional 3D US image using appropriate rendering techniques.

The real-time visualization of the tissue features annotated on the pre-interventional 3D MRI image on the N^{th} interventional 3D US image provides visual guidance during the intervention. For example, in the case of an ablation procedure, the visualization of tumor boundaries on the real-time US image can assist in accurately positioning the ablation device and monitoring the progress of the ablation process. Similarly, in the case of a biopsy procedure, the visualization of lesion boundaries can aid in precisely targeting the biopsy needle and ensuring that the desired tissue sample is obtained.

The method 600 overcomes the computational limitations of conventional rigid or analytical deformation calculations, which are not conducive to real-time applications due to their extensive computational demands. By leveraging the trained MR-US deformation model and the trained US-US deformation model, the method 600 enables real-time visualization of tissue features during interventions, enhancing the accuracy and efficiency of the procedures.

In an alternative embodiment, the operations 608, 610, 612, and 614 may be repeated multiple times throughout the intervention, with each iteration using the most recently acquired interventional 3D US image as the input for operation 608. This iterative approach allows for continuous updating of the registered 3D MRI image, ensuring that the tissue features annotated on the pre-interventional 3D MRI image are accurately overlaid on the interventional 3D US image, even as the anatomical region of interest undergoes motion or deformation due to factors such as patient movement, respiratory motion, or interactions with interventional devices.

Overall, the method 600 provides a robust and efficient approach for real-time visualization of tissue features during interventional procedures, leveraging the complementary strengths of MRI and ultrasound imaging modalities. By combining the high-resolution anatomical information from pre-interventional MRI data with the real-time imaging capabilities of ultrasound, the method 600 enhances the guidance and precision of interventional procedures, ultimately leading to improved patient outcomes.

Referring to FIG. 7, a flowchart of a method 700 for acquiring and storing imaging data for training MR-US and US-US deformation models is shown. The method 700 provides a systematic approach for acquiring and organizing the necessary imaging data for the self-supervised training of the deformation models.

The method 700 begins at operation 702, where a three-dimensional 3D MRI image of an imaging subject is acquired in a selected position. The selected position may correspond to a specific pose or orientation of the imaging subject, such as supine, decubitus, or any other position relevant to the planned interventional procedure and the anatomical region of interest.

In one embodiment, the 3D MRI image is acquired using a gated or breath-hold imaging technique to minimize artifacts caused by respiratory motion or other patient movement during the image acquisition process. This ensures that the acquired 3D MRI image accurately represents the anatomical structures in the selected position without substantial distortions or blurring due to motion. In another embodiment, the 3D MRI image is acquired with the administration of a contrast agent to enhance the visualization of specific tissues or anatomical structures of interest.

At operation 704, a plurality of 3D US images of the imaging subject are acquired in the selected position from operation 702, capturing multiple respiratory states. These 3D US images may be obtained using a 3D ultrasound probe or transducer.

In one embodiment, the plurality of 3D US images are acquired at a predetermined imaging frequency over a predetermined duration of time, capturing the anatomical motion and deformation of the region of interest due to respiration. This approach allows for the collection of a comprehensive set of US images representing the full range of respiratory states, which enables training of robust deformation models able to account for respiratory-induced anatomical deformations.

In another embodiment, the plurality of 3D US images are acquired while the imaging subject is instructed to hold their breath at different respiratory states (e.g., full inspiration, full expiration, and intermediate states). This approach ensures that the acquired US images accurately represent specific respiratory phases, enabling the deformation models to learn the transformations associated with each respiratory state.

At operation 706, the 3D MRI image acquired in operation 702 and the plurality of 3D US images of the imaging subject acquired in operation 704 are stored in a first training data set for the MR-US deformation model. This training data set will be used to train the MR-US deformation model to accurately register and align the MRI images with the corresponding US images, accounting for the anatomical deformations and pose variations captured in the US images. In one embodiment, the 3D MRI image and the plurality of 3D US images are stored in a structured database or file system, where each MRI image is associated with the corresponding set of US images acquired in the same selected position and respiratory states. This organization facilitates efficient retrieval and processing of the training data during the model training process. In another embodiment, the 3D MRI image and the plurality of 3D US images are stored along with metadata describing the imaging parameters, patient information, and other relevant details. This metadata can be used for data management, quality control, and potential future refinements or extensions of the deformation models.

At operation 708, the plurality of 3D US images of the imaging subject acquired in operation 704 are stored in a second training data set for the US-US deformation model. This training data set will be used to train the US-US deformation model to compute the transformations between pairs of US images representing different respiratory states for the same patient. In one embodiment, the plurality of 3D US images are stored in a structured database or file system, where each set of US images acquired in a specific selected position and respiratory states is organized together. This organization facilitates efficient retrieval and processing of the training data during the model training process for the US-US deformation model. In another embodiment, the plurality of 3D US images are stored along with metadata describing the imaging parameters, patient information, respiratory state annotations, and other relevant details. This metadata can be used for data management, quality control, and potential future refinements or extensions of the US-US deformation model.

By executing the operations of method 700, the imaging data for training both the MR-US and US-US deformation models is acquired and organized in a structured manner. Method 700 ensures that the deformation models are trained on a comprehensive set of imaging data, capturing various patient positions, respiratory states, and anatomical deformations, enabling accurate and robust performance during real-time multimodal deformable image registration for image-guided interventions.

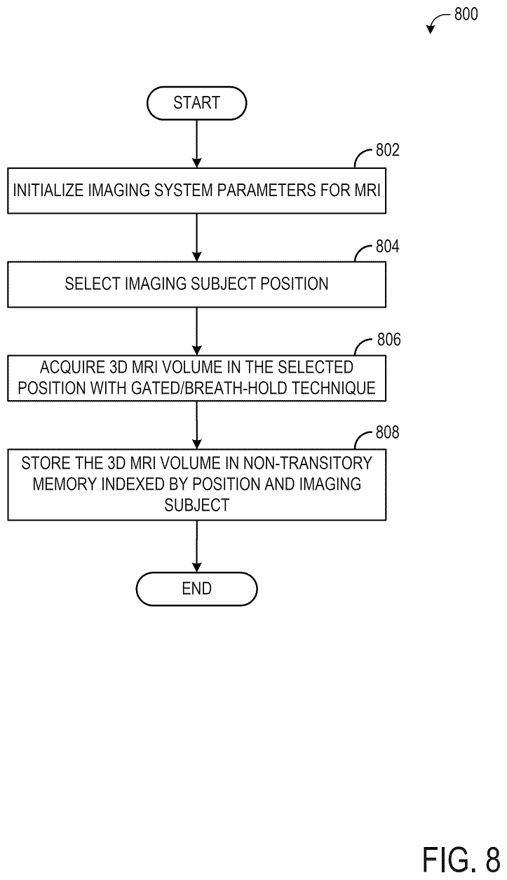

Referring to FIG. 8, a flowchart of a method 800 for acquiring and storing a 3D MRI image for use in training an MR-US deformation model is shown. The method 800 outlines the steps involved in acquiring and storing a 3D MRI image of an imaging subject, which will be used as part of the training data for the MR-US deformation model.

The method 800 begins at operation 802, where the imaging system parameters for MRI acquisition are initialized. In one embodiment, this operation involves configuring the MRI imaging device 240 (shown in FIG. 2) to acquire a 3D MRI image with specific imaging parameters tailored for the anatomical region of interest and the imaging subject. These parameters may include, but are not limited to, the field of view (FOV), spatial resolution, slice thickness, imaging sequence (e.g., T1-weighted, T2-weighted, or other sequences), and contrast agent administration (if applicable).

At operation 804, the imaging subject's position is selected. The selection of the imaging subject's position determines the orientation and pose of the anatomical structures captured in the 3D MRI image. In one embodiment, the imaging subject is positioned in a supine position. In another embodiment, the imaging subject may be positioned in a decubitus position, which may be preferred for certain anatomical regions or interventional procedures.

At operation 806, the 3D MRI image is acquired in the selected position using a gated or breath-hold technique. The gated or breath-hold technique is employed to minimize artifacts caused by respiratory motion or other patient movement during the image acquisition process. In one embodiment, the 3D MRI image is acquired using a gated technique, where the MRI data acquisition is synchronized with the imaging subject's respiratory cycle. This technique involves acquiring MRI data at specific phases of the respiratory cycle, such as end-expiration or end-inspiration, to capture the anatomical structures in a consistent state of motion or deformation.

In another embodiment, the 3D MRI image is acquired using a breath-hold technique, where the imaging subject is instructed to hold their breath for a short period of time (typically 10-20 seconds) during the MRI data acquisition. This technique eliminates respiratory motion artifacts by capturing the anatomical structures in a static state during the breath-hold period.

At operation 808, the acquired 3D MRI image is stored in non-transitory memory, such as the non-transitory memory 206 of the image processing system 200 (shown in FIG. 2). The 3D MRI image is indexed or associated with metadata that includes the imaging subject's position and other relevant information, such as the imaging subject's identifier, anatomical region of interest, and imaging parameters used during acquisition.

In one embodiment, the 3D MRI image is stored in a hierarchical file structure, where each 3D MRI image is stored in a subdirectory or folder associated with the imaging subject's identifier and the specific position or pose in which the image was acquired. This file structure may be organized based on patient identifiers, imaging session timestamps, or other relevant metadata to facilitate efficient retrieval and management of the 3D MRI images.

In another embodiment, the 3D MRI image is stored in a database or file system accessible by the image processing system responsible for training the MR-US deformation model. The association between the 3D MRI image and the imaging subject's position may be implemented using data structures such as linked lists, hash tables, or other suitable data organization techniques.

Following operation 808, the method 800 may end. The acquired and stored 3D MRI image, along with its associated metadata, will be used as part of the training data for the MR-US deformation model, which is responsible for registering or aligning the MRI image with corresponding ultrasound images during the image-guided intervention process.

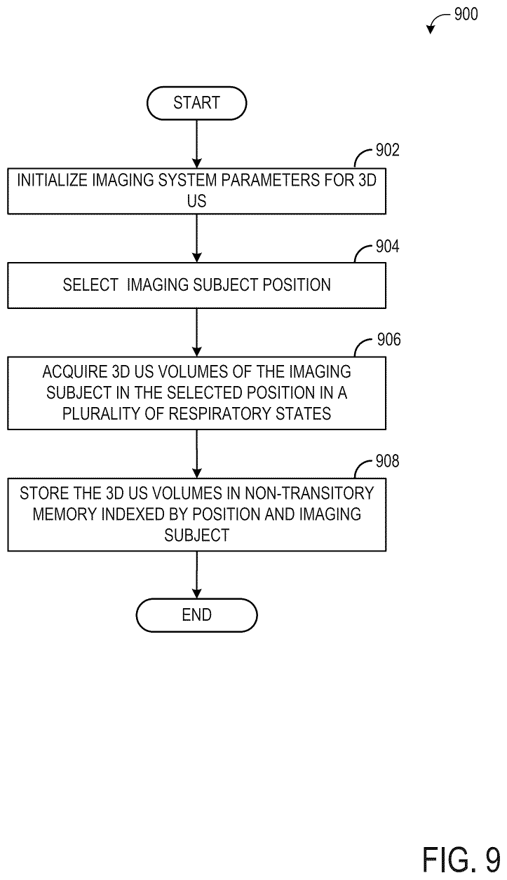

Referring to FIG. 9, a flowchart of a method 900 for acquiring and storing 3D US images of an imaging subject in various respiratory states, for use in training a US-US deformation model, is shown. The method 900 is designed to capture the anatomical motion and deformation of the imaging subject's internal anatomy due to respiration, which is used for training the US-US deformation model to accurately register US images acquired during different respiratory states.

The method 900 begins at operation 902, where the imaging system parameters for 3D US imaging are initialized. In one embodiment, this operation involves configuring the US imaging device 260 (shown in FIG. 2) to acquire 3D US images. This may include setting the imaging mode to 3D or 4D (real-time 3D) mode, adjusting the imaging depth and field of view to encompass the anatomical region of interest, and optimizing imaging parameters such as gain, dynamic range, and frequency to ensure high-quality image acquisition. In another embodiment, operation 902 may involve initializing a simultaneous MR and ultrasound imaging system with a 3D US probe. This integrated imaging system allows for the simultaneous acquisition of MRI and US data, which may facilitate the subsequent registration of the two modalities during the training process.

At operation 904, the position or pose of the imaging subject is selected. In one embodiment, the imaging subject may be positioned in a supine position, which is a common starting point for many interventional procedures. In another embodiment, the imaging subject may be positioned in a decubitus position, which may be more suitable for certain anatomical regions or interventional approaches. The selection of the imaging subject's position at operation 904 may be based on the specific anatomical region of interest and the planned interventional procedure. For example, if the region of interest is the liver or abdomen, the imaging subject may be positioned in a left or right decubitus position to provide better access and visualization of the target anatomy.

At operation 906, 3D US images of the imaging subject are acquired in the selected position, capturing a plurality of respiratory states. In one embodiment, the 3D US images are acquired continuously at a predetermined imaging frequency over a predetermined duration of time, allowing for the capture of multiple respiratory cycles and the associated anatomical motion and deformation. In another embodiment, the 3D US images are acquired while the imaging subject is instructed to hold their breath at different respiratory states, such as full inspiration, full expiration, and intermediate states. This approach may provide a more controlled and reproducible set of respiratory states, which can be beneficial for training the US-US deformation model. During the acquisition of the 3D US images at operation 906, the imaging subject's respiratory state may be monitored using various techniques, such as respiratory bellows or optical tracking systems. This information can be used to associate each acquired 3D US image with a specific respiratory state, enabling accurate labeling of the training data for the US-US deformation model.

At operation 908, the acquired 3D US images are stored in non-transitory memory, indexed by the imaging subject's position and respiratory state. In one embodiment, the 3D US images are stored in a hierarchical file structure, where each position or pose of the imaging subject is represented by a separate directory or folder. Within each position directory, the 3D US images are organized and labeled according to the corresponding respiratory state, such as “inspiration,” “expiration,” or specific lung image measurements. In another embodiment, the 3D US images are stored in a database or data management system, where each image is associated with metadata describing the imaging subject, position, and respiratory state. This metadata can be used to efficiently retrieve and organize the training data for the US-US deformation model, as well as for subsequent analysis or quality control purposes. The storage of the 3D US images at operation 908 may also involve preprocessing steps, such as image enhancement, noise reduction, or data compression, to optimize the storage and retrieval of the training data. Additionally, the stored 3D US images may be used in conjunction with other imaging modalities, such as MRI or CT, to create multimodal training datasets for the US-US deformation model or other deformation models used in the image registration pipeline.

By acquiring and storing 3D US images in various respiratory states and positions, the method 900 provides a comprehensive dataset for training the US-US deformation model. This model, in turn, plays a role in the real-time registration of interventional US images with pre-interventional US and MRI data, enabling accurate visualization of tissue features and enhancing the guidance and precision of image-guided interventions.

Referring to FIG. 10, a block diagram of an MR-US deformation model training process 1000 is shown, illustrating the training of a deep learning-based deformation model for registering MRI images to US images. The MR-US deformation model training process 1000 is designed to learn the complex, non-linear deformations required to align MRI and US data, accounting for the inherent differences in image appearance and characteristics between these two imaging modalities.

The training process 1000 begins with an MRI image 1002, which is a 3D or 4D MRI image of an imaging subject. In one embodiment, the MRI image 1002 is a pre-interventional, high-resolution 3D MRI image acquired using an MRI imaging device, such as the MRI apparatus 10 shown in FIG. 1. The MRI image 1002 provides detailed anatomical information.

The MRI image 1002 is input into an MRI feature extractor 1004, which is responsible for extracting relevant features from the MRI image 1002. In one embodiment, the MRI feature extractor 1004 is a CNN that applies a series of convolutional, pooling, and non-linear activation operations to the MRI image 1002, producing a feature map that captures the salient anatomical and structural information present in the MRI image 1002. The MRI feature extractor 1004 may be pre-trained on a large dataset of MRI images to learn a robust set of features that are effective for various medical imaging tasks.

In parallel, the training process 1000 also receives a US image 1006, which is a 3D or 4D US image of the same imaging subject as the MRI image 1002. In one embodiment, the US image 1006 is a 3D US image acquired using a US imaging device, such as the US imaging device 260 shown in FIG. 2. The US image 1006 captures the anatomical region of interest in a different modality, providing complementary information to the MRI image 1002.

The US image 1006 is input into a US feature extractor 1008, which is responsible for extracting relevant features from the US image 1006. Similar to the MRI feature extractor 1004, the US feature extractor 1008 may be a CNN that applies a series of convolutional, pooling, and non-linear activation operations to the US image 1006, producing a feature map that captures the salient anatomical and structural information present in the US image 1006. The US feature extractor 1008 may be pre-trained on a large dataset of US images to learn a robust set of features specific to the US imaging modality.

The feature maps produced by the MRI feature extractor 1004 and the US feature extractor 1008 are concatenated to form a concatenated MRI and US feature map 1010. In one embodiment, the concatenation operation involves stacking the feature maps along the channel dimension, creating a single feature map that combines the information from both the MRI and US modalities.

The concatenated MRI and US feature map 1010 is then input into a deformation field generator 1012, which is responsible for predicting the deformation field required to align the MRI image 1002 with the US image 1006. In one embodiment, the deformation field generator 1012 is a CNN or a fully connected neural network that takes the concatenated MRI and US feature map 1010 as input and outputs a dense deformation field, also known as a warp field 1014.

The warp field 1014 represents the displacement or deformation vectors that map each voxel (3D pixel) in the MRI image 1002 to its corresponding location in the US image 1006. The warp field 1014 captures the complex, non-linear deformations required to account for the differences in image appearance, patient positioning, and anatomical deformations between the MRI and US modalities.

The warp field 1014 is then applied to the MRI image 1002 using a spatial transformer 1016, which is a differentiable module that warps or deforms the input image based on the provided deformation field. In one embodiment, the spatial transformer 1016 applies the warp field 1014 to the MRI image 1002 using a sampling kernel, such as bilinear or trilinear interpolation, to produce a deformed MRI image 1018 that is aligned with the US image 1006.

The deformed MRI image 1018 and the US image 1006 are then compared using a similarity metric 1020, which is a measure of the alignment or registration quality between the two images. In one embodiment, the similarity metric 1020 is a normalized cross-correlation or mutual information metric, which evaluates the similarity between the deformed MRI image 1018 and the US image 1006 without requiring ground truth deformation fields or manual annotations.

The similarity metric 1020 is used to update the parameters of the MRI feature extractor 1004, the US feature extractor 1008, and the deformation field generator 1012 through backpropagation, with the goal of maximizing the similarity between the deformed MRI image 1018 and the US image 1006. This iterative training process allows the MR-US deformation model to learn the complex deformations required to register MRI and US data, without the need for manually annotated ground truth deformation fields, which can be time-consuming and challenging to obtain.

In an alternative embodiment, the training process 1000 may incorporate additional components or modules to enhance the performance of the MR-US deformation model. For example, the training process 1000 may include a regularization module that applies constraints or penalties to the deformation field generator 1012, encouraging the model to learn smooth and physically plausible deformations. Additionally, the training process 1000 may include a multi-scale or pyramid approach, where the MRI and US images are processed at multiple resolutions, allowing the model to capture both coarse and fine-grained deformations.

The MR-US deformation model training process 1000 is designed to be incremental and adaptive, allowing the model to be trained on new data as it becomes available. In one embodiment, the training process 1000 initializes the weights of the MRI feature extractor 1004, the US feature extractor 1008, and the deformation field generator 1012 using pre-trained weights from a previous training session or a publicly available model. As new MRI and US image pairs are acquired, the training process 1000 can be re-executed, fine-tuning the model parameters to adapt to the specific characteristics of the new data. Overall, the MR-US deformation model training process 1000 provides a robust and efficient approach for learning the complex deformations required to register MRI and US data, enabling accurate and real-time visualization of tissue features during image-guided interventions.

Referring to FIG. 11, a flowchart of a method 1100 for training an MR-US deformation model to deformably register 3D MRI images with 3D US images from the same imaging subject is shown. The method 1100 is designed to train a deep learning-based deformation model that can accurately align and register MRI and ultrasound image data, accounting for the inherent differences in image appearance and characteristics between these two imaging modalities. Further, method 1100 employs a novel self-supervised training approach which enables training on large amounts of data by bypassing the need to train on labeled data, which is a recognized bottleneck in the field of machine learning.

The method 1100 begins at operation 1102, where an MRI image and a US image from the same imaging subject are selected from a training dataset. In one embodiment, the training dataset comprises a collection of pre-interventional 3D MRI images and corresponding 4D US images acquired for a set of patients or imaging subjects. The 4D US images capture the anatomical region of interest in multiple respiratory states and poses, providing a comprehensive representation of the deformations and variations that may occur during an interventional procedure. In an alternative embodiment, the training dataset may be initialized with data from a single patient or imaging subject, and incrementally expanded as additional patient data becomes available. This approach allows the MR-US deformation model to be trained and refined iteratively, leveraging the model weights from previous training sessions as a starting point for subsequent training with new data.

At operation 1104, features are extracted from the MRI image using an MRI feature extractor to produce an MRI feature map. The MRI feature extractor is a component of the MR-US deformation model designed to capture relevant anatomical and structural information from the MRI image, encoding it into a compact feature representation or feature map. In one embodiment, the MRI feature extractor may be pre-trained on a large dataset of MRI images, allowing it to learn general features and patterns that are characteristic of MRI data. During the training of the MR-US deformation model, the weights of the MRI feature extractor may be fine-tuned to adapt to the specific anatomical region and imaging characteristics of the training dataset.

At operation 1106, features are extracted from the US image using a US feature extractor to produce a US feature map. Similar to the MRI feature extractor, the US feature extractor is a component of the MR-US deformation model designed to capture relevant anatomical and structural information from the US image, encoding it into a compact feature representation or feature map. In one embodiment, the US feature extractor may be pre-trained on a large dataset of ultrasound images, allowing it to learn general features and patterns that are characteristic of ultrasound data. During the training of the MR-US deformation model, the weights of the US feature extractor may be fine-tuned to adapt to the specific anatomical region and imaging characteristics of the training dataset.

At operation 1108, the MRI feature map and the US feature map are concatenated to produce a concatenated MRI and US feature map. This concatenation operation combines the feature representations extracted from the MRI and US images, enabling the subsequent components of the MR-US deformation model to learn the relationships and correspondences between the two modalities. In an alternative embodiment, instead of concatenating the feature maps, the MRI feature map and the US feature map may be combined using other fusion techniques, such as element-wise addition, element-wise multiplication, or more complex fusion operations implemented as separate neural network layers.

At operation 1110, the concatenated MRI and US feature map is mapped to a warp field using a deformation field generator. The deformation field generator takes the concatenated feature map as input and outputs a warp field, which represents the deformation or displacement field required to align the MRI image with the corresponding US image. In one embodiment, the deformation field generator may be designed to output a dense warp field, where each voxel in the MRI image is associated with a displacement vector. In another embodiment, the deformation field generator may output a sparse warp field, where the displacements are defined only at a subset of control points, and the displacements for the remaining voxels are interpolated from the control point displacements.

At operation 1112, the warp field produced by the deformation field generator is applied to the MRI image using a spatial transformer to produce a deformed MRI image. The spatial transformer is a differentiable module that applies the warp field to the MRI image using a sampling kernel, such as bilinear or trilinear interpolation. This operation effectively warps or deforms the MRI image to align it with the corresponding US image, based on the deformation field predicted by the deformation field generator. In an alternative embodiment, instead of using a spatial transformer, the warp field may be applied to the MRI image using a non-rigid registration algorithm, such as B-spline or free-form deformation. This approach may provide additional flexibility and control over the deformation process, but may also introduce additional computational complexity.

At operation 1114, a loss is determined based on a similarity between the deformed MRI image and the US image. The loss function quantifies the degree of misalignment or dissimilarity between the deformed MRI image and the corresponding US image. In one embodiment, the loss function may be based on a similarity metric, such as normalized cross-correlation or mutual information, which measures the degree of alignment between the deformed MRI image and the US image. In another embodiment, the loss function may be based on a combination of similarity metrics and regularization terms, such as smoothness constraints or sparsity constraints on the deformation field.

At operation 1116, the parameters of the spatial transformer, the deformation field generator, the US feature extractor, and the MRI feature extractor are updated based on the loss determined in operation 1114. This update operation may be performed using an optimization algorithm, such as stochastic gradient descent or one of its variants, which adjusts the parameters of the various components of the MR-US deformation model in a direction that minimizes the loss function. In one embodiment, the update operation may be performed using backpropagation, where the gradients of the loss function with respect to the parameters of the various components are computed and used to update the parameters in the opposite direction of the gradients, scaled by a learning rate. In another embodiment, the update operation may be performed using more advanced optimization techniques, such as momentum-based methods or adaptive learning rate methods, which can improve the convergence speed and stability of the training process.

The operations 1102 through 1116 are repeated for multiple iterations, using different pairs of MRI and US images from the training dataset. This iterative process allows the MR-US deformation model to gradually learn the complex relationships and deformations required to accurately register MRI and ultrasound data, by minimizing the loss function over a diverse set of training examples. In one embodiment, the training process may be performed in a batch mode, where a mini-batch of MRI and US image pairs is processed at each iteration, and the gradients of the loss function are accumulated over the mini-batch before updating the parameters of the MR-US deformation model. In another embodiment, the training process may be performed in an online mode, where the parameters of the MR-US deformation model are updated after processing each individual pair of MRI and US images, potentially leading to faster convergence but also increased computational overhead.

The method 1100 for training the MR-US deformation model is designed to leverage the power of deep learning and unsupervised learning techniques to address the challenging problem of multimodal deformable image registration. By learning directly from the data, without the need for manually annotated ground truth deformation fields, the MR-US deformation model can adapt to the specific characteristics and variations present in the training dataset, enabling accurate and robust registration of MRI and ultrasound data for image-guided interventions.

Referring to FIG. 12, a block diagram of a US-US deformation model training process 1200 is shown. This process is designed to train a deep learning-based deformation model that can accurately compute the transformation between pairs of US images representing different respiratory states or poses for the same patient. The trained US-US deformation model enables the registration of pre-interventional US images with interventional US images acquired during the procedure.

The training process 1200 takes as input a moving US image 1202 and a fixed US image 1204. These images are typically 3D US images acquired during the pre-interventional stage, capturing the anatomical region of interest in different respiratory states or poses. The moving US image 1202 represents the image that needs to be deformed or warped to align with the fixed US image 1204, which serves as the reference or target image.

The moving US image 1202 and the fixed US image 1204 are fed into a deformation field generator 1206, which is a deep learning model responsible for predicting the deformation field required to align the moving US image 1202 with the fixed US image 1204. The deformation field generator 1206 may be based on various deep learning architectures, such as CNNs, recurrent neural networks (RNNs), or transformer models, depending on the specific requirements and characteristics of the US image data. In one embodiment, the deformation field generator 1206 is a CNN-based model that takes the moving US image 1202 and the fixed US image 1204 as input and outputs a dense deformation field, where each voxel in the moving US image 1202 is assigned a displacement vector that maps it to the corresponding location in the fixed US image 1204.

In another embodiment, the deformation field generator 1206 is a transformer-based model that leverages self-attention mechanisms to capture long-range dependencies and global context in the US images. This approach may be particularly useful for handling large deformations and complex anatomical motions that can occur between different respiratory states or poses. The deformation field generator 1206 outputs a warp field 1208, which represents the predicted deformation field that maps the voxels of the moving US image 1202 to their corresponding locations in the fixed US image 1204.

The warp field 1208 is then applied to the moving US image 1202 by a spatial transformer 1210, which is a differentiable module that performs the warping or deformation operation using techniques such as bilinear or trilinear interpolation. The spatial transformer 1210 produces a deformed moving US image 1212, which is an approximation of the moving US image 1202 after being deformed to align with the fixed US image 1204.

The deformed moving US image 1212 and the fixed US image 1204 are then compared using a similarity metric 1214, which measures the degree of alignment or similarity between the two images. In one embodiment, the similarity metric 1214 is based on normalized cross-correlation, which measures the similarity between the intensity patterns of the two images. A higher cross-correlation value indicates better alignment between the deformed moving US image 1212 and the fixed US image 1204. In another embodiment, the similarity metric 1214 is based on mutual information, which measures the statistical dependence between the intensity distributions of the two images. A higher mutual information value suggests that the deformed moving US image 1212 and the fixed US image 1204 are well-aligned and share similar intensity patterns.

The training process 1200 for the US-US deformation model comprises an unsupervised training process that employs a similarity metric 1214 to evaluate the alignment between pairs of US images, such as the moving US image 1202 and the fixed US image 1204. The similarity metric 1214 is calculated without the use of labeled ground truth data or manually annotated deformation fields. Instead, the similarity metric 1214 measures the degree of alignment or similarity between the deformed moving US image 1212 and the fixed US image 1204 after applying the warp field predicted by the deformation field generator 1206. The similarity metric 1214 is used as a loss function or objective function during the training process, where the parameters of the deformation field generator 1206 are adjusted to maximize the similarity between the deformed moving US image 1212 and the fixed US image 1204.

The training process 1200 is typically performed using a large dataset of US image pairs, representing different respiratory states and poses for multiple patients. The dataset may be augmented with various data augmentation techniques, such as random cropping, rotation, or intensity transformations, to improve the generalization capability of the trained US-US deformation model.

Once the training process 1200 is complete, the trained US-US deformation model, which includes the deformation field generator 1206 and the spatial transformer 1210, can be used during the interventional phase to register pre-interventional US images with interventional US images acquired in real-time. This registration process enables the visualization of tissue features annotated on the pre-interventional US images in the context of the interventional US images, providing valuable guidance for the interventional procedure.

Referring to FIG. 13, a flowchart of a method 1300 for training a US-US deformation model to deformably register US images in multiple states of deformation from the same imaging subject, is shown. The method 1300 may be employed by an image processing system to train the US-US deformation model using a training dataset comprising pairs of US images representing different respiratory states or poses for the same patient.

At operation 1302, the image processing system selects a moving US image and a fixed US image from the same imaging subject from a training dataset. The training dataset comprises a plurality of pairs of US images, wherein each pair includes a moving US image and a fixed US image. The moving US image and the fixed US image represent the same anatomical region of the imaging subject but captured in different states of deformation, such as different respiratory phases or different patient poses. In one embodiment, the moving US image and the fixed US image are selected from a set of pre-interventional 4D US images acquired for the imaging subject. The 4D US images capture the anatomical region of interest over time, allowing for the extraction of individual 3D US images representing different respiratory states or poses. The moving US image and the fixed US image may be selected from different time points within the 4D US image, ensuring that they represent distinct states of deformation.

At operation 1304, the image processing system maps the moving US image and the fixed US image to a warp field using a deformation field generator. The deformation field generator is a component of the US-US deformation model responsible for estimating the deformation or transformation required to align the moving US image with the fixed US image. In one embodiment, the deformation field generator is a deep learning-based model, such as a CNN or a vision transformer. The deformation field generator takes the moving US image and the fixed US image as inputs and outputs a warp field that describes the deformation or displacement field required to map the voxels (3D pixels) of the moving US image to their corresponding locations in the fixed US image.

At operation 1306, the image processing system applies the warp field to the moving US image using a spatial transformer to produce a deformed moving US image. The spatial transformer is a component of the US-US deformation model responsible for applying the warp field estimated by the deformation field generator to the moving US image. In one embodiment, the spatial transformer is a differentiable module that applies the warp field to the moving US image using a sampling kernel, such as bilinear or trilinear interpolation. This allows the spatial transformer to be integrated into the deep learning pipeline and trained end-to-end with the other components of the US-US deformation model. In another embodiment, the spatial transformer applies the warp field to the moving US image using a non-rigid registration algorithm, such as B-spline or free-form deformation. This approach may provide more accurate deformations but may be computationally more expensive than the differentiable spatial transformer approach.

At operation 1308, the image processing system determines a loss based on a similarity between the deformed moving US image and the fixed US image. The loss represents a measure of the dissimilarity or misalignment between the deformed moving US image and the fixed US image, and is used to guide the training of the US-US deformation model. In one embodiment, the loss is calculated using a similarity metric, such as normalized cross-correlation, mutual information, or structural similarity index. The similarity metric is applied to the deformed moving US image and the fixed US image, and the resulting value is used as the loss. A higher value of the similarity metric indicates a better alignment between the two images, and therefore a lower loss. In another embodiment, the loss is calculated using a combination of multiple similarity metrics, where each metric is weighted based on its importance or relevance to the specific application or anatomical region of interest. For example, in regions with high contrast and well-defined boundaries, a metric such as normalized cross-correlation may be weighted more heavily, while in regions with low contrast or diffuse boundaries, a metric such as mutual information may be given a higher weight.

At operation 1310, the image processing system updates the parameters of the spatial transformer and the deformation field generator based on the loss. This operation is part of the training process for the US-US deformation model, where the model's parameters are adjusted to minimize the loss and improve the alignment between the deformed moving US image and the fixed US image. In one embodiment, the update of the parameters is performed using a gradient-based optimization algorithm, such as stochastic gradient descent (SGD) or Adam. The gradients of the loss with respect to the parameters of the spatial transformer and the deformation field generator are computed using backpropagation, and the parameters are updated in the direction that minimizes the loss. In another embodiment, the update of the parameters is performed using a meta-learning approach, where the US-US deformation model is trained to learn how to update its own parameters based on the loss and the input data. This approach can lead to faster convergence and better generalization performance, particularly when the training data is limited or exhibits significant variability.

The method 1300 may be repeated for multiple iterations, using different pairs of moving US images and fixed US images from the training dataset. This iterative process allows the US-US deformation model to learn the complex deformations and transformations required to align US images representing different respiratory states or poses for the same patient. After training, the US-US deformation model can be used in conjunction with a trained MR-US deformation model to enable real-time visualization of tissue features annotated on pre-interventional MRI images during interventional procedures guided by real-time US imaging. The US-US deformation model is responsible for aligning the pre-interventional US images with the interventional US images, while the MR-US deformation model is responsible for aligning the pre-interventional MRI images with the pre-interventional US images.

FIG. 14 illustrates the efficacy of the disclosed systems and methods for real-time visualization of tissue features annotated on pre-interventional MRI images during an interventional procedure guided by US imaging. FIG. 14 presents three images: a pre-interventional MRI image 1402, an interventional 3D US image 1404, and a visualization 1406 of the pre-interventional MRI features overlaid on the interventional 3D US image.

The pre-interventional MRI image 1402 is a high-resolution, 3D image image acquired prior to the interventional procedure. This image provides superior soft tissue contrast and lesion conspicuity, enabling accurate delineation and annotation of tissue features of interest, such as organ boundaries or tumor boundaries. In the example shown in FIG. 14, the pre-interventional MRI image 1402 depicts a cross-sectional view of an anatomical region, with a well-defined lesion or tumor visible within the tissue.

Prior to the intervention, the pre-interventional MRI image 1402 undergoes a process of annotation, where the tissue features of interest, in this case, the lesion or tumor boundaries, are manually or automatically segmented and delineated. These annotated tissue features are then registered with a plurality of pre-interventional 3D US images, capturing the anatomical region in various respiratory states and poses, using a trained MR-US deformation model. This registration process produces a series of deformed 3D MRI images, each corresponding to a specific pre-interventional 3D US image.

During the interventional procedure, an interventional 3D US image 1404 is acquired in real-time using a 3D US probe or transducer. This interventional 3D US image 1404 provides a live view of the anatomical region, enabling real-time guidance and monitoring of the interventional procedure. However, due to the inherent limitations of US imaging, such as poor soft tissue contrast and lesion conspicuity, the lesion or tumor boundaries may not be clearly visible in the interventional 3D US image 1404.

To overcome this limitation and enhance the visualization of tissue features during the intervention, the disclosed systems and methods employ a trained US-US deformation model to register the interventional 3D US image 1404 with a corresponding pre-interventional 3D US image from the plurality of pre-interventional 3D US images. This registration process determines a warp field that describes the transformation required to align the pre-interventional 3D US image with the interventional 3D US image 1404, accounting for any anatomical motion or deformation that may have occurred between the acquisition of the two images.

The warp field is then applied to the deformed 3D MRI image corresponding to the registered pre-interventional 3D US image, effectively aligning the pre-interventional MRI data with the interventional US data. This alignment process results in the visualization 1406, where the tissue features annotated on the pre-interventional MRI image 1402, such as the lesion or tumor boundaries, are overlaid onto the interventional 3D US image 1404.

In the visualization 1406, the annotated tissue features from the pre-interventional MRI image 1402 are integrated with the real-time interventional 3D US image 1404, providing the clinician with a comprehensive view of the anatomical region and the tissue features of interest. This visualization enables precise guidance and targeting during the interventional procedure, as the clinician can clearly identify the lesion or tumor boundaries, even in the presence of poor soft tissue contrast or lesion conspicuity in the US image alone.

The overlay of the pre-interventional MRI features on the interventional 3D US image 1406 is achieved in real-time, overcoming the computational limitations of conventional rigid or analytical deformation calculations. By leveraging the trained US-US deformation model and the pre-computed deformed 3D MRI images, the disclosed systems and methods enable real-time visualization of tissue features during interventions, enhancing the accuracy and efficiency of the procedures.

FIG. 15 illustrates the efficacy of the currently disclosed approach for registration of a moving US image 1502 to a fixed US image 1504, resulting in a registered image 1506. The moving US image 1502 represents an image that is to be deformed or warped to align with the fixed US image 1504, which serves as a reference or target image. In the context of the present disclosure, the moving US image 1502 may correspond to a pre-interventional 3D US image acquired prior to an intervention, while the fixed US image 1504 may represent an interventional 3D US image acquired during the intervention.

The moving US image 1502 and the fixed US image 1504 depict the same anatomical region of interest, but may exhibit differences due to factors such as patient motion, respiratory motion, or changes in the positioning of the imaging probe. These differences can lead to misalignment between the two images, making it challenging to directly compare or integrate the information they contain.

To address this challenge, the present disclosure employs a trained US-US deformation model, as described in previous sections, to register the moving US image 1502 to the fixed US image 1504. The US-US deformation model is a deep learning-based model that has been trained to compute the transformation required to align pairs of US images representing different respiratory states or poses for the same patient.

During the registration process, the trained US-US deformation model takes the moving US image 1502 and the fixed US image 1504 as inputs and outputs a warp field that describes the deformation or displacement field required to align the moving US image 1502 with the fixed US image 1504. This warp field accounts for any anatomical motion or deformation that may have occurred between the acquisition of the two images, such as changes due to respiration, patient movement, or interventional device placement.

The warp field is then applied to the moving US image 1502 using a spatial transformer or a non-rigid registration algorithm, such as B-spline or free-form deformation. This process deforms or warps the moving US image 1502, effectively aligning it with the fixed US image 1504, resulting in the registered image 1506.

The registered image 1506 represents the moving US image 1502 after it has been deformed or warped to match the fixed US image 1504. In the registered image 1506, the anatomical structures and features present in the moving US image 1502 are now aligned with their corresponding structures and features in the fixed US image 1504, as highlighted by the white circle in each of the three images, enabling direct comparison and integration of information between the two images.

It is important to note that while FIG. 15 depicts the registration of US images, the present disclosure is not limited to this specific imaging modality. The principles and techniques described herein can be extended to other imaging modalities, such as magnetic resonance imaging (MRI) or computed tomography (CT), by training appropriate deformation models tailored to the specific modalities involved.

The disclosure also provides support for a method comprising: acquiring a pre-interventional three-dimensional (3D) image of an imaging subject in a first imaging modality, wherein the first imaging modality is one of magnetic resonance imaging (MRI), computed tomography (CT), and positron emission tomography (PET), acquiring a first interventional 3D ultrasound (US) image of the imaging subject during an intervention, registering the pre-interventional 3D image in the first imaging modality with the first interventional 3D US image using a first trained deformation model to produce a first deformed 3D image in the first imaging modality, acquiring a subsequent interventional 3D US image during the intervention, registering the first 3D US image with the subsequent interventional 3D US image using a trained US-US deformation model to determine a warp field, applying the warp field to the first deformed 3D image in the first imaging modality to produce a 3D image in the first imaging modality registered to the subsequent interventional 3D US image, and visualizing tissue features annotated on the pre-interventional 3D image in real-time on the subsequent interventional 3D US image using the 3D image in the first imaging modality registered to the subsequent interventional 3D US image. In a first example of the method, the first imaging modality is MRI, and the first trained deformation model is a trained MR-US deformation model. In a second example of the method, optionally including the first example, the trained MR-US deformation model is trained by initializing weights of the MR-US deformation model with training data from a single patient, and iteratively re-training the MR-US deformation model with additional patient data, wherein each subsequent training session utilizes the weights from a previous training session, and wherein the training data includes gated or breath-hold 3D MRI images and multiple 3D US images representing different respiratory states and poses. In a third example of the method, optionally including one or both of the first and second examples, visualizing tissue features annotated on the pre-interventional 3D image in the first imaging modality on the subsequent interventional 3D US image comprises overlaying the 3D image in the first imaging modality registered to the subsequent interventional 3D US image onto the subsequent interventional 3D US image. In a fourth example of the method, optionally including one or more or each of the first through third examples, registering the pre-interventional 3D image in the first imaging modality with the first interventional 3D US image using the first trained deformation model, or registering the first interventional 3D US image with the subsequent interventional 3D US image using the trained US-US deformation model, further comprises utilizing features derived from the pre-interventional 3D image in the first imaging modality and the first interventional 3D US image, including radiomics features and Gaussian Mixture Model tissue class probabilities. In a fifth example of the method, optionally including one or more or each of the first through fourth examples, the trained US-US deformation model is trained using pre-interventional 3D US images from the imaging subject, captured at a pre-determined imaging frequency over a pre-determined duration of time, to capture multiple respiratory states. In a sixth example of the method, optionally including one or more or each of the first through fifth examples, acquiring the pre-interventional 3D image of the imaging subject in the first imaging modality and acquiring the first interventional 3D US image of the imaging subject are performed using a simultaneous imaging system capable of acquiring images in the first imaging modality and ultrasound images concurrently.

The disclosure also provides support for an image processing system comprising: a display device, a non-transitory memory including instructions, and a processor, wherein, when executing the instructions, the processor causes the image processing system to: acquire a pre-interventional three-dimensional (3D) magnetic resonance imaging (MRI) image of an imaging subject, and a plurality of pre-interventional 3D ultrasound (US) images capturing multiple respiratory states and poses of the imaging subject, register the pre-interventional 3D MRI image with each of the plurality of pre-interventional 3D US images using a trained MR-US deformation model to produce a plurality of deformed 3D MRI images, acquire an interventional 3D US image during an intervention, register a pre-interventional 3D US image from the plurality of pre-interventional 3D US images with the interventional 3D US image using a trained US-US deformation model to determine a warp field, apply the warp field to a deformed 3D MRI image from the plurality of deformed 3D MRI images corresponding to the pre-interventional 3D US image to produce a 3D MRI image registered to the interventional 3D US image, and display, via the display device, tissue features annotated on the pre-interventional 3D MRI image on the interventional 3D US image using the 3D MRI image registered to the interventional 3D US image during the intervention. In a first example of the system, the system further comprises a simultaneous MR and ultrasound imaging system with a 3D US probe configured to acquire the pre-interventional 3D MRI image and the plurality of pre-interventional 3D US images. In a second example of the system, optionally including the first example, the trained MR-US deformation model and the trained US-US deformation model are incrementally trained using network model weights initialized from previous training sessions with data from the imaging subject. In a third example of the system, optionally including one or both of the first and second examples, the processor further causes the image processing system to annotate tissue features on the pre-interventional 3D MRI image, wherein the tissue features include organ or tumor boundaries. In a fourth example of the system, optionally including one or more or each of the first through third examples, the trained MR-US deformation model is trained by initializing weights of the MR-US deformation model with training data from a single patient, and iteratively re-training the MR-US deformation model with additional patient data, wherein each subsequent training session utilizes the weights from a previous training session, and wherein the training data includes gated or breath-hold 3D MRI images and multiple 3D US images representing different respiratory states and poses. In a fifth example of the system, optionally including one or more or each of the first through fourth examples, the trained US-US deformation model is trained using pre-interventional 3D US images from the imaging subject, captured at a pre-determined imaging frequency over a pre-determined duration of time, to capture multiple respiratory states. In a sixth example of the system, optionally including one or more or each of the first through fifth examples, the processor further causes the image processing system to visualize tissue features annotated on the pre-interventional 3D MRI image on the interventional 3D US image by overlaying the registered 3D MRI image onto the interventional 3D US image.

The disclosure also provides support for a method for training deformation models for multimodal image registration in image-guided interventions, the method comprising: acquiring a pre-interventional three-dimensional (3D) magnetic resonance imaging (MRI) image and a plurality of pre-interventional four-dimensional (4D) ultrasound (US) images representing different respiratory states for a patient, initializing model weights for an MR-US deformation model and a US-US deformation model using data from at least one previously scanned patient, training the MR-US deformation model to register MR images to US images using the pre-interventional 3D MRI image and the plurality of pre-interventional 4D US images, wherein the plurality of pre-interventional 4D US images capture patient-specific anatomical motion due to respiration and interventional device placement, training the US-US deformation model to compute transformations between pairs of US images representing different respiratory states for the patient, and registering a pre-interventional MRI image with real-time US images acquired during an intervention using the trained MR-US deformation model and the trained US-US deformation model. In a first example of the method, training the MR-US deformation model comprises an unsupervised training process that employs a similarity metric to evaluate alignment of the MR images to the US images, the similarity metric calculated without use of labeled ground truth data. In a second example of the method, optionally including the first example, the similarity metric includes one or more of normalized cross-correlation, mutual information, or structural similarity index, and wherein the unsupervised training process adjusts parameters of the MR-US deformation model based on the similarity metric determined across the plurality of pre-interventional 4D US images and the pre-interventional 3D MRI image. In a third example of the method, optionally including one or both of the first and second examples, training the US-US deformation model comprises an unsupervised training process that employs a similarity metric to evaluate alignment between pairs of US images, the similarity metric calculated without use of labeled ground truth data. In a fourth example of the method, optionally including one or more or each of the first through third examples, the similarity metric includes one or more of normalized cross-correlation, mutual information, or structural similarity index, and wherein the unsupervised training process adjusts parameters of the US-US deformation model based on the similarity metric determined across a plurality of pairs of the pre-interventional 4D US images. In a fifth example of the method, optionally including one or more or each of the first through fourth examples, the method further comprises: annotating tissue features, including organ or tumor boundaries, on the pre-interventional MRI image, and using the trained MR-US deformation model and the trained US-US deformation model to transfer the annotated organ or tumor boundaries to the real-time US images acquired during the intervention.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method comprising:
acquiring a pre-interventional three-dimensional (3D) image of an imaging subject in a first imaging modality, wherein the first imaging modality is one of magnetic resonance imaging (MRI), computed tomography (CT), and positron emission tomography (PET);
acquiring a first interventional 3D ultrasound (US) image of the imaging subject during an intervention;
registering the pre-interventional 3D image in the first imaging modality with the first interventional 3D US image using a first trained deformation model to produce a first deformed 3D image in the first imaging modality;
acquiring a subsequent interventional 3D US image during the intervention;
registering the first interventional 3D US image with the subsequent interventional 3D US image using a trained US-US deformation model to determine a warp field;
applying the warp field to the first deformed 3D image in the first imaging modality to produce a 3D image in the first imaging modality registered to the subsequent interventional 3D US image; and
visualizing tissue features annotated on the pre-interventional 3D image in real-time on the subsequent interventional 3D US image using the 3D image in the first imaging modality registered to the subsequent interventional 3D US image.

2. The method of claim 1, wherein the first imaging modality is MRI, and the first trained deformation model is a trained MR-US deformation model.

3. The method of claim 2, wherein the trained MR-US deformation model is trained by initializing weights of the MR-US deformation model with training data from a single patient, and iteratively re-training the MR-US deformation model with additional patient data, wherein each subsequent training session utilizes the weights from a previous training session, and wherein the training data includes gated or breath-hold 3D MRI images and multiple 3D US images representing different respiratory states and poses.

4. The method of claim 1, wherein visualizing tissue features annotated on the pre-interventional 3D image in the first imaging modality on the subsequent interventional 3D US image comprises overlaying the 3D image in the first imaging modality registered to the subsequent interventional 3D US image onto the subsequent interventional 3D US image.

5. The method of claim 1, wherein registering the pre-interventional 3D image in the first imaging modality with the first interventional 3D US image using the first trained deformation model, or registering the first interventional 3D US image with the subsequent interventional 3D US image using the trained US-US deformation model, further comprises utilizing features derived from the pre-interventional 3D image in the first imaging modality and the first interventional 3D US image, including radiomics features and Gaussian Mixture Model tissue class probabilities.

6. The method of claim 1, wherein the trained US-US deformation model is trained using pre-interventional 3D US images from the imaging subject, captured at a pre-determined imaging frequency over a pre-determined duration of time, to capture multiple respiratory states.

7. The method of claim 1, wherein acquiring the pre-interventional 3D image of the imaging subject in the first imaging modality and acquiring the first interventional 3D US image of the imaging subject are performed using a simultaneous imaging system capable of acquiring images in the first imaging modality and ultrasound images concurrently.

8. An image processing system comprising:
a display device;
a non-transitory memory including instructions; and
a processor, wherein, when executing the instructions, the processor causes the image processing system to:
acquire a pre-interventional three-dimensional (3D) magnetic resonance imaging (MRI) image of an imaging subject, and a plurality of pre-interventional 3D ultrasound (US) images capturing multiple respiratory states and poses of the imaging subject;
register the pre-interventional 3D MRI image with each of the plurality of pre-interventional 3D US images using a trained MR-US deformation model to produce a plurality of deformed 3D MRI images;
acquire an interventional 3D US image during an intervention;
register a pre-interventional 3D US image from the plurality of pre-interventional 3D US images with the interventional 3D US image using a trained US-US deformation model to determine a warp field;
apply the warp field to a deformed 3D MRI image from the plurality of deformed 3D MRI images corresponding to the pre-interventional 3D US image to produce a 3D MRI image registered to the interventional 3D US image; and
display, via the display device, tissue features annotated on the pre-interventional 3D MRI image on the interventional 3D US image using the 3D MRI image registered to the interventional 3D US image during the intervention.

9. The image processing system of claim 8, wherein the system further comprises a simultaneous MR and ultrasound imaging system with a 3D US probe configured to acquire the pre-interventional 3D MRI image and the plurality of pre-interventional 3D US images.

10. The image processing system of claim 8, wherein the trained MR-US deformation model and the trained US-US deformation model are incrementally trained using network model weights initialized from previous training sessions with data from the imaging subject.

11. The image processing system of claim 8, wherein the processor further causes the image processing system to annotate tissue features on the pre-interventional 3D MRI image, wherein the tissue features include organ or tumor boundaries.

12. The image processing system of claim 8, wherein the trained MR-US deformation model is trained by initializing weights of the MR-US deformation model with training data from a single patient, and iteratively re-training the MR-US deformation model with additional patient data, wherein each subsequent training session utilizes the weights from a previous training session, and wherein the training data includes gated or breath-hold 3D MRI images and multiple 3D US images representing different respiratory states and poses.

13. The image processing system of claim 8, wherein the trained US-US deformation model is trained using pre-interventional 3D US images from the imaging subject, captured at a pre-determined imaging frequency over a pre-determined duration of time, to capture multiple respiratory states.

14. The image processing system of claim 8, wherein the processor further causes the image processing system to visualize tissue features annotated on the pre-interventional 3D MRI image on the interventional 3D US image by overlaying the registered 3D MRI image onto the interventional 3D US image.

15. A method for training deformation models for multimodal image registration in image-guided interventions, the method comprising:
acquiring a pre-interventional three-dimensional (3D) magnetic resonance imaging (MRI) image and a plurality of pre-interventional four-dimensional (4D) ultrasound (US) images representing different respiratory states for a patient;
initializing model weights for an MR-US deformation model and a US-US deformation model using data from at least one previously scanned patient;
training the MR-US deformation model to register MR images to US images using the pre-interventional 3D MRI image and the plurality of pre-interventional 4D US images, wherein the plurality of pre-interventional 4D US images capture patient-specific anatomical motion due to respiration and interventional device placement;
training the US-US deformation model to compute transformations between pairs of US images representing different respiratory states for the patient; and
registering a pre-interventional MRI image with real-time US images acquired during an intervention using the trained MR-US deformation model and the trained US-US deformation model.

16. The method of claim 15, wherein training the MR-US deformation model comprises an unsupervised training process that employs a similarity metric to evaluate alignment of the MR images to the US images, the similarity metric calculated without use of labeled ground truth data.

17. The method of claim 16, wherein the similarity metric includes one or more of normalized cross-correlation, mutual information, or structural similarity index, and wherein the unsupervised training process adjusts parameters of the MR-US deformation model based on the similarity metric determined across the plurality of pre-interventional 4D US images and the pre-interventional 3D MRI image.

18. The method of claim 15, wherein training the US-US deformation model comprises an unsupervised training process that employs a similarity metric to evaluate alignment between pairs of US images, the similarity metric calculated without use of labeled ground truth data.

19. The method of claim 18, wherein the similarity metric includes one or more of normalized cross-correlation, mutual information, or structural similarity index, and wherein the unsupervised training process adjusts parameters of the US-US deformation model based on the similarity metric determined across a plurality of pairs of the pre-interventional 4D US images.

20. The method of claim 15, further comprising annotating tissue features, including organ or tumor boundaries, on the pre-interventional MRI image, and using the trained MR-US deformation model and the trained US-US deformation model to transfer the annotated organ or tumor boundaries to the real-time US images acquired during the intervention.
